(19) United States
(12) Patent Application Publication
(10) Pub. No.: US 2015/0182583 A1
(43) Pub. Date: Jul. 2, 2015

(54) METHODS FOR TREATMENT AND PREVENTION OF TAUOPATHIES BY INHIBITING ENDOTHELIN RECEPTORS

(71) Applicant: Board of Regents, The University of Texas System, Austin, TX (US)

(72) Inventor: Isaiah J. Fidler, Houston, TX (US)

(73) Assignee: Board Of Regents, The University Of Texas System, Austin, TX (US)

(21) Appl. No.: 14/419,807

(22) PCT Filed: Aug. 6, 2013
(86) PCT No.: PCT/US2013/053873

§ 371 Date: Feb. 5, 2015

Related U.S. Application Data
(60) Provisional application No. 61/679,850, filed on Aug. 6, 2012.

(51) Int. Cl. A61K 38/12; A61K 38/06; A61K 45/06; A61K 38/08

(52) U.S. Cl. CPC A61K 38/12; A61K 38/08; A61K 38/06; A61K 45/06

(57) ABSTRACT

Compositions and methods of treatment of tauopathies are provided. In some embodiments, an antagonist of endothclin receptor A (ETA) and endothelin receptor B (ETB) may be administered to a subject to reduce tau production or accumulation, e.g., in astrocytes. The antagonist may be a dual ETA and ETB receptor antagonist. In some aspects, methods are provided for the treatment of chemo-brain, hypoxia, brain ischemia, surgical dementia, glioblastoma, or a traumatic brain injury (TBI).

This application claims the benefit of U.S. Provisional Patent Application No. 61/679,580, filed on Aug. 6, 2012, the entirety of which is incorporated herein by reference.

### BACKGROUND OF THE INVENTION

1. Field of the Invention

The present invention relates generally to the fields of molecular biology and medicine. More particularly, it concerns methods of treating tauopathies.

2. Description of Related Art

Astrocytes are cells in the brain which play an important role in homeostasis. Astrocyte functions include transporting nutrients from blood to neurons, helping to protect neurons, participating in neuronal signal transmission, and maintaining homeostasis of local ion concentrations and pH. Astrocytes are central to the catabolism of selected amino acids in the brain, as well as to the synthesis of new amino acids. Additionally, astrocytes can modulate synaptic function via affects on glutamate transporters, which convey glutamate from the synaptic cleft into the cell, and communication between astrocytes can occur via ATP release and binding to purine receptors on adjacent astrocytes. Gap junctions can also contribute to an astrocyte syncytium for the exchange of small molecules and cell-cell communication (Simard and Nedergaard, 2004).

Astrocytes are also implicated in various disease states. For example, astrocytes can upregulate survival genes in tumor cells and induce protection from chemotherapy (Kim et al., 2011). Astrocytes can also play a role in brain microenvironment that can affect brain metastases (Fidler, 2011). It has only relatively recently been proposed that astrocytes may play a role in the development or progression of neurodegenerative diseases, e.g., via alteration of glutaminergic synaptic transmission resulting in excitotoxicity or from alteration of function as a result of interactions with amyloid-β (Maragakis and Rothstein, 2006).

One of the hallmark features of Alzheimer's disease (AD) and other tauopathies is the accumulation of tau protein in neurons and glia. This pattern contrasts markedly with the normal CNS distribution, in which tau is expressed predominantly in axons, and is only expressed at low levels in oligodendrocytes and astrocytes. To assess the contribution of astrocytes to tauopathies, transgenic mice were generated in which the tau protein was expressed selectively in astrocytes. In these mice, there was abundant astrocyte tau pathology associated with neuronal staining of phosphorylated neurofilament epitopes, axon degeneration, and inclusion formation, all of which indicated neuron injury; however, no significant neuronal loss was observed (Forman et al., 2005). In an extension of these initial observations, investigators developed transgenic mice overexpressing the tauP301L mutation—which is linked to frontotemporal dementia and parkinsonism (FTDP) in humans—in astrocytes. These mice developed neuromuscular abnormalities with loss of strength. The astrocyte tau pathology was also associated with a reduction in expression and function of the astrocyte-specific glutamate transporters GLT1 and GLAST (Dabir et al., 2006). The selective tau expression in astrocytes in these models provides more evidence of an astrocyte-mediated effect in models of dementia.

Tauopathies continue to be a problem for many patients despite advances in the understanding of these diseases. Clearly, there is a need for new methods to treat tauopathies.

### SUMMARY OF THE INVENTION

The present invention overcomes limitations in the prior art by providing methods for treating or delaying the onset of a tauopathy such as, for example, chemo brain (e.g., resulting from administration of paclitaxel or temozolide), a traumatic brain injury (TBI), hypoxia, brain ischemia (cerebral ischemia), stroke, or surgical dementia. In various aspects, an endothelin receptor antagonist, such as a dual endothelin receptor antagonist, may be therapeutically administered to a subject, such as a human patient, to treat or delay the onset of a tauopathy. In some embodiments, tau production in astrocytes can be reduced in a subject by administration of an endothelin receptor antagonist.

An aspect of the present invention relates to a method for delaying the onset or progression of a tauopathy in a subject comprising administering to the subject: (a) an amount of a dual endothelin receptor antagonist effective to inhibit endothelin receptor A and endothelin B receptor; or (b) an effective amount of an endothelin receptor A antagonist and an endothelin receptor B antagonist. Another aspect of the present invention relates to a composition comprising: (a) a dual endothelin receptor antagonist effective to inhibit endothelin receptor A and endothelin B receptor; or (b) an endothelin receptor A antagonist and an endothelin receptor B antagonist; for use in delaying the onset or progression of a tauopathy in a subject. In some preferred embodiments, the tauopathy is not Alzheimer's disease. Nonetheless, in some embodiments, a dual endothelin receptor antagonist (or administration of both an inhibitor of ETA and an inhibitor of ETB) may be used to reduce the onset of, prevent, or slow the progression of Alzheimer's disease. In some embodiments, the dual endothelin receptor antagonist is administered to the subject. In some embodiments, the dual endothelin receptor antagonist or endothelin receptor antagonist may inhibit endothelin receptor A and/or endothelin receptor B phosphorylation. The dual endothelin receptor antagonist may be PD145065, TAK-044, tezosentan, or bosentan(4-tert-butyl-N-[6-(2-hydroxyethoxy)-5-(2-methoxyphenoxy)-2-(pyrimidin-2-yl)pyrimidin-4-yl]benzene-1-sulfonamide). The endothelin receptor antagonist may be an endothelin receptor A antagonist, and wherein the method comprises administering an effective amount of a endothelin receptor B antagonist. The endothelin receptor A antagonist may be BQ123. The endothelin receptor B antagonist may be PD143296 or BQ788. The endothelin receptor A antagonist and the endothelin receptor B antagonist may be administered in a single formulation or separately. The endothelin receptor antagonist or dual endothelin receptor antagonist may be a peptide antagonist, such as, e.g., TAK-044. The endothelin receptor antagonist may be comprised in a liposome, such as, e.g., a CNS targeted liposome. The endothelin receptor antagonist may further comprise a central nervous system (CNS) targeting agent. The CNS targeting agent may be a polypeptide. The CNS targeting polypeptide may be bound to the endothelin receptor antagonist or may be comprised in fusion protein with the endothelin receptor antagonist.

In some embodiments, the tauopathy is amyotrophic lateral sclerosis/parkinsonism-dementia complex, argyrophilic grain dementia, corticobasal degeneration, creutzfeldt-Jakob disease, dementia pugilistica, diffuse neurofibrillary tangles with calcificationa, Down's syndrome, frontotemporal dementia with parkinsonism (linked to chromosome 17), Gerstmann-Straussler-Scheinker disease, Hallervorden-Spatz disease, myotonic dystrophy, Niemann-Pick disease (type C), non-Guamanian motor neuron disease with neurofibrillary tangles, Pick's disease, frontotemporal dementia and parkinsonism (FTDP), postencephalitic parkinsonism, prion protein cerebral amyloid angiopathy, progressive subcortical gliosis, progressive supranuclear palsy, subacute sclerosing panencephalitis, tangle only dementia, cognitive disorder, hypoxia, brain ischemia (cerebral ischemia), stroke, or surgical dementia, glioblastoma or glioblastoma multiforme (GBM), a traumatic brain injury (TBI), chronic encephalopathy, brain trauma, dementia pugilistica, or chemo-brain (e.g., resulting from administration of paclitaxel and/or temozolomide). The method may be further defined as a method for delaying the onset of a tauopathy in a subject. The subject may be at risk for developing a tauopathy. The subject may comprise a gene mutation associated with a tauopathy or may comprise a family history of tauopathy. In some embodiments, the subject has reduced cognitive or memory function. In some embodiments, the subject has been diagnosed with a tauopathy. The subject may be a human. In some embodiments, the amount of the endothelin receptor antagonist administered to the subject is from about 10 mg/kg to about 150 mg/kg. The endothelin receptor antagonist may be administered orally, intravenously, topically, intradermally, intraarterially, intraperitoneally, intracranially, intrathecally, intracerebroventricularly, mucosally, intrarectally (suppository), intraocularally or subcutaneously. The method may further comprise administering a second therapeutic agent to the subject. The second therapeutic agent may be an acetylcholinesterase inhibitor or an anti-inflammatory compound.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

Chemo brain, also called “chemo-fog,” “chemo-brain,” or “chemotherapy-related cognitive impairment or cognitive dysfunction,” refers to the cognitive changes that can occur as a side effect of chemotherapy. These changes may be temporary changes in memory and the thinking process. Chemo-brain typically involves one or more of the following symptoms: difficulty concentrating and thinking clearly, difficulty in multi tasking, decreased memory, shortened attention span, feelings of disorganization and/or difficulties concentrating. Chemo brain may result from a wide variety of chemotherapeutics. In some embodiments, chemo brain results from administration of paclitaxel or temozolomide. As shown in the below examples, administration of paclitaxel or temozolomide to mice bearing brain cancer or to normal mice was observed to result in the production of TAU, and this production of TAU was observed to be prevented or treated by the administration a dual endothelin receptor antagonist.

“Subject” as used herein can refer to mammals, such as mice, rats, rabbits, goats, cats, dogs, cows, apes and humans.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein, the specification “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Hypothesis of endothelin involvement in tau production in astrocytes.

FIG. 2: Altered expression of genes in astrocytes.

FIG. 3: Increased endothelin-1 in astrocytes cultured in MDA231. Increased expression of endothelin-1 was observed.

FIG. 4: Astrocytes are GFAP-positive after brain hypoxia.

FIG. 5: ETAR and ETBR expression level in Astrocytes.

FIG. 6: Production of endothelin 1 or 2 by astrocytes cultured under normoxia vs. hypoxia (Real-time PCR).

FIG. 7: ET-1 and ET-2 expression level in Astrocytes. Cells were treated with IL-6 (5 ng/ml), IL-8 (5 ng/ml), VEGF (5 ng/ml), and ET-1 (1 μM) for 24 hr.

FIG. 8: Expression of TAU and APO(E) by astrocytes cultured under hypoxia vs. normoxia using Real-time PCR.

FIG. 9: Production of endothelins by brain endothelial cells cultured under normoxia vs. hypoxia using Real-time PCR.

FIG. 10: Expression of APO(E) and TAU by brain endothelial cells cultured under normoxia vs. hypoxia using Real-time PCR.

FIG. 11: Expression level of APO(E) and TAU expression level in control astrocytes and astrocytes treated with BQ123 and /or BQ788. Cells were treated with ET-1 (1 μM) for 24 hr; cells were treated with BQ123(100 nM) and/or BQ788(100 nM) for 2 hr prior to treatment with ET1 where indicated.

FIG. 12: Expression level of APOE and TAU expression level in control astrocytes and astrocytes treated with BQ123 plus PQ788 or PD145065. Treatment groups were as follows: Control, ET-1 (100 nM), ET-1+BQ123/BQ788 (1 μM), ET-1+PD145065 (1 μM). Cells were treated with ET-1 for 24 hr. BQ123, BQ788 or PD145065 was added 2 hrs prior to ET-1 stimulation.

FIG. 13: TAU expression under normal and hypoxic conditions.

FIG. 14: Methodology to identify genes whose expression is altered by BQ123 and BQ788.

FIG. 15: MDA231 Protection Assay using PD165045.

FIG. 16: Immunohistochemisty of GFAP and TAU expression after brain trauma using an in vivo mouse model of brain trauma Immunofluorescent staining of GFAP (left) or GFAP and TAU (right) are shown. Astrocytes were observed to express GFAP and TAU.

FIG. 17: Schedule of PD 145065 pre-treatment wound and harvest of brain tissue.

FIG. 18: Immunohistochemistry results from acute brain hypoxia experiments. Brains from mice with acute brain hypoxia contrasted with brains of control mice kept in normoxia. As shown in the figure, a large increase in TAU-positive astrocytes was observed in brains having been exposed to the acute brain hypoxia. TAU expression is shown as white dots.

FIG. 19: Photo of Hypoxia Chamber.

FIG. 20: Immunohistochemistry for TAU expression. TAU is shown as white dots. A significant decrease in TAU expression was observed in brain of mice treated with PD145065.

FIG. 21: IHC of brains from mice bearing LN229 GBM treated with paclitaxel or TMZ. TAU is shown as white dots.

FIG. 22: Body weight of control mice or mice treated with paclitaxel alone or paclitaxel and PD 145065. Note loss of weight in paclitaxel-treated mice.

FIG. 23: Prevention and/or treatment of paclitaxel toxicity by PD145065 (PD).

### DESCRIPTION OF ILLUSTRATIVE EMBODIMENTS

The present invention is based in part on the identification that endothelin antagonists can be used to treat a tauopathy. In some embodiments, a dual endothelin receptor A (ETA) and endothelin receptor B (ETB) antagonist may be used to reduce tau accumulation or production. In other embodiments, a selective ETA antagonist may be administered to a subject in combination with an ETB antagonist to reduce tau accumulation or production.

### I. TAUOPATHIES

Tauopathies are characterized by CNS accumulation of tau protein aggregates known as tangles. Tau normally serves to bind and stabilize neuronal microtubules, to facilitate their roles in cellular structure, polarity and transport (Stamer et al., 2002). Recent work suggests that tau plays a beneficial role in supporting normal hippocampal memory-related function (Boekhoorn et al., 2006). Phosphorylation can disrupt these activities and promote cytoskeletal destabilization (Sengupta et al., 1998). Aberrantly phosphorylated forms of tau aggregate into PHFs, and these into insoluble neurofibrillary tangles (NFTs), a feature associated with tauopathies such as AD (Kopke et al., 1993). In some embodiments, the tauopathy is not Alzheimer's disease. In other embodiments, methods and compositions of the present invention may be used to treat, prevent or slow the onset of, or reduce or inhibit the progression of Alzheimer's disease.

Tau isoforms are single gene products that differ by the inclusion of inserts in an N-terminal projection domain and tandem repeats within a C-terminal microtubule-binding domain (Goedert et al., 1989). Whereas human tau is normally phosphorylated at 2-3 moles/mole of protein, PHF-tau from AD brain is hyperphosphorylated at a 7-10 molar ratio (Kopke et al., 1993).

### II. ENDOTHELIN ANTAGONISTS

Endothelins are a family of small peptides (i.e., ET-1, ET-2, and ET-3) that initiate signaling through the g-protein coupled receptors: endothelin receptor A (ETA) and endothelin receptor B (ETB). Endothelins were originally identified as potent vasoconstrictors, but may play a role in cell signaling, apoptosis, bone remodeling, metastasis, and/or angiogenesis (Nelson et al., 2003).

In some aspects of the present invention, an endothelin receptor antagonist may be used to therapeutically treat a tauopathy. Endothelin receptor antagonists generally selectively inhibit endothelin A (ETA) receptor and/or endothelin B (ETB) receptor. The ETB may be endothelin B receptor type 1 (ETB 1) or endothelin B receptor type 2 (ETB2). As shown in the below examples, endothelin receptor antagonists can be used to decrease tau protein accumulation and/or production.

Endothelin antagonists include, e.g., PD143296 (Ac-D-Phe-L-Leu-L-Phe-L-Ile-L-Ile-L-Trp.2Na; SEQ ID NO:1) and PD145065 (Ac-[(R)-2-10, 11-dihydro-5H-dibenzo[a,d]cyclohepten-5-yl]Gly)-L-Leu-L-Asp-L-Ile-L-Ile-L-Trp.2Na; the peptide portion is listed as SEQ ID NO:2), BQ123 (CAS Number 136553-81-6), BQ788 (CAS Number 156161-89-6), BMS182874, TAK-044 (Takeda), atrasentan, tezosentan, sitaxsentan, enrasentan, BMS-207940 (BristolMyers Squibb), BMS-193884, J-104132 (Banyu Pharmaceutical), VML 588/Ro 61-1790 (Vanguard Medica), T-0115 (Tanabe Seiyaku), YM-598, LU 135252, A-127722, ABT-627, A-192621, A-182086, TBC3711, BSF208075, S-0139, TBC2576, TBC3214, PD156707, PD180988, ABT-546, ABT-627, Z1611, RPR118031A, SB247083, SB217242, S-Lu302872, TPC10950, SB209670, and PD145065. Additional useful endothelin antagonists can be found in U.S. Patent Application Publication No. US 2002/0082285, incorporated herein by reference.

PD145065 (CAS No. 153049-49-1) is also referred to as N-Acetyl-α-[10,11-Dihydro-5H-dibenzo[a,d]cycloheptadien-5-yl]-D-Gly-Leu-Asp-Ile-Ile-Trp (SEQ ID NO:2) or AC-DBHG-LEU-ASP-ILE-ILE-TRP;AC-D-BHG-LEU-ASP-ILE-ILE-TRP-OH (SEQ ID NO:2), and has the following structure:

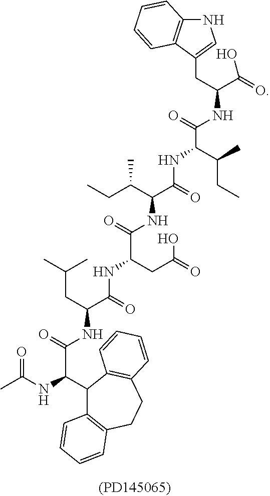 [SMILES: CC[C@H](C)[C@H](NC(=O)[C@H](CC(=O)O)NC(=O)[C@H](CC(C)C)NC(=O)[C@H](NC(C)=O)C1c2ccccc2CCc2ccccc21)C(=O)N[C@H](C(=O)N[C@@H](Cc1c[nH]c2ccccc12)C(=O)O)[C@@H](C)CC]

Some endothelin antagonists selectively antagonize ETA receptors. These selective ETA antagonists include, e.g., ABT-627, BQ123, and BMS182874. BQ123 is a specific endothelin A antagonist, and is the sodium salt of cyclo(-D-Trp-D-Asp-Pro-D-Val-Leu-) (SEQ ID NO:3). The structure of BQ123 is:

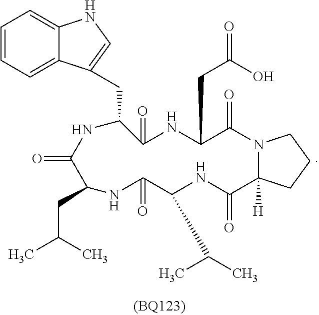 [SMILES: CC(C)C[C@@H]1NC(=O)[C@@H](C(C)C)NC(=O)[C@@H]2CCCN2C(=O)[C@@H](CC(=O)O)NC(=O)[C@@H](Cc2c[nH]c3ccccc23)NC1=O]

Some endothelin antagonists selectively antagonize ETB receptors. These selective ETA antagonists include, e.g., A-192621, PD143296, and BQ788. BQ-788 is a specific endothelin B antagonist, and is the sodium salt of N-cis-2,6-dimethylpiperidinocarbony-1-L-gamma-methylleucyl-D-1methoxycarbonyl triptophanyl-DNIe (see Ishikawa et al., 1994). The structure of BQ788 is:

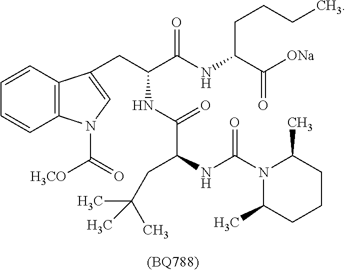

Dual endothelin receptor antagonists may be used in various aspects of the present invention. A dual endothelin receptor antagonist is any compound which selectively antagonizes both ETA and ETB receptors. Dual endothelin receptor antagonists include, e.g., PD145065, TAK-044, tezosentan (N-[6-(2-Hydroxyethoxy)-5-(2-methoxyphenoxy)-2-[2-(2H-tetrazol-5-yl)pyridin-4-yl]pyrimidin-4-yl]-5-propan-2-ylpyridine-2-sulfonamide), etc. In some embodiments, the dual endothelin receptor antagonist is bosentan (4-tert-butyl-N-[6-(2-hydroxyethoxy)-5-(2-methoxyphenoxy)-2-(pyrimidin-2-yl)pyrimidin-4-yl]benzene-1-sulfonamide).

In various embodiments, it may also be possible to indirectly inhibit endothelin receptors by reducing the synthesis of endothelin. Thus, in addition to a conventional endothelin antagonist, a compound that inhibits the formation of endogenous endothelin also can be used decrease the activity of endothelin receptors. One class of such compounds is the endothelin converting enzyme (ECE) inhibitors. It is anticipated that, in various aspects of the present invention, an ECE inhibitor may be used to produce a therapeutic effect in the treatment of a tauopathy. ECE inhibitors include, e.g., CGS34225 (i.e., N-((1-((2(S)-(acetylthio)-1-oxopentyl)-amino)-1-cyclopentyl)-carbonyl-S 4-phenylphenyl-alanine methyl ester) and phosphoramidon (i.e., N-(a-rhamnopyranosyloxyhydroxyphosphinyl)-Leu-Trp).

The pharmaceutical compositions include those wherein the endothelin antagonists are administered in an effective amount to achieve their intended purpose. More specifically, a “therapeutically effective amount” means an amount effective to ameliorate, eliminate, or retard the progression of a tauopathy. Determination of a therapeutically effective amount is within the capability of those skilled in the art, especially in light of the detailed disclosure provided herein.

In some embodiments, an endothelin antagonist may be administered orally to a subject, such as a human patient in a dose from about 10 to about 200 mg daily for an average adult patient (70 kg), e.g., divided into two to three doses per day. For example, individual tablets or capsules contain about 0.1 to about 50 mg endothelin antagonist, in a suitable pharmaceutically acceptable vehicle or carrier, for administration in single or multiple doses, once or several times per day. For example, bosentan may be administered in an oral formulation in an amount of from about 25-150 mg, about 50-150 mg or about 62.5-125 mg per dosage. In some embodiments, bosentan may be administered twice daily for about 4 weeks. In some embodiments, the dosage of bosentan may be from about 0.5-5 mg/kg, 1-4 mg/kg, 1-2 mg/kg, or 2-4 mg/kg. Dosages for intravenous, buccal, or sublingual administration may, e.g., range from about 0.1 to about 10 mg/kg per single dose as required. In some embodiments of the present invention, the dose of an endothelin antagonist may range from about 10 mg/kg to about 150 mg/kg, or any range derivable therein. In practice, the physician determines the actual dosing regimen that is most suitable for an individual patient, and the dosage varies with the age, weight, and response of the particular patient. The above dosages are exemplary of the average case, but there can be individual instances in which higher or lower dosages are merited, and such are within the scope of this invention. In some embodiments, an ETA inhibitor and an ETB inhibitor may be administered to a subject. The ETA inhibitor and the ETB inhibitor may be administered simultaneously (e.g., in the same pharmaceutical preparation) or sequentially. The ETA inhibitor and the ETB inhibitor may be administered to a subject within a period of less than 1 minute, or 1, 2, 3, 4, 5, 5-10, 10-30, 30-60, 1-60 minutes, or 1, 2, 3, 4, 5, 6, 7, 8, 9, 10, 11, 12, 13, 14, 15, 16, 17, 18, 19, 20, 21, 22, 23, or 24 hours or any range derivable therein. In some embodiments, it may be preferable to administer the ETA inhibitor and the ETB inhibitor to the subject substantially simultaneously or within a period of less than 12 hours.

The actual dosage amount of a composition of the present invention administered to a subject can be determined by physical and physiological factors such as body weight, severity of condition, the type of disease being treated (i.e., type of tauopathy), previous or concurrent therapeutic interventions, idiopathy of the patient and on the route of administration. The practitioner responsible for administration will, in any event, determine the concentration of active ingredient(s) in a composition and appropriate dose(s) for the individual subject.

Pharmaceutical compositions of the present invention comprise an effective amount of one or more endothelin antagonist or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one endothelin antagonist or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, targeting agents (e.g., CNS targeting agents), lubricants, sweetening agents, flavoring agents, gels (e.g., gelatin), dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The endothelin antagonist may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, intrabucally (e.g., in a suppository), locally, via inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, see, for example, Remington: The Science and Practice of Pharmacy, 21s^{t} Ed. Lippincott Williams and Wilkins, 2005, incorporated herein by reference).

Pharmaceutical compositions may comprise, for example, at least about 0.1% of an endothelin antagonist. In some embodiments, the an active compound may comprise between about 2% to about 75% of the weight of the unit, or from about 25% to about 60%, for example, and any range derivable therein.

The composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. In the case of proteinacious compositions of the invention or endothelin receptor antagonists that comprise a peptide, it may also be preferable that the action of proteases be inhibited during storage of compositions. This can be accomplished by the additional of protease inhibitors and/or the storage of the compositions at low temperature prior to administration.

In embodiments where pharmaceutical compositions are provided in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof In some embodiments, an isotonic agent such as, e.g., a sugar, sodium chloride, or combinations thereof may be included.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In certain embodiments, an oral composition may comprise one or more binders, excipients, disintegration agents, lubricants, flavoring agents, and combinations thereof In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In some embodiments, suppositories may be formed from mixtures containing, for example, an endothelin receptor antagonist in the range of about 0.5% to about 10%, or from about 1% to about 2%.

A. Dosages

Endothelin of the invention will generally be used in an amount effective to achieve the intended purpose. For use to treat or delay the onset or progression of a tauopathy, the molecules of the invention, or pharmaceutical compositions thereof, are administered in a therapeutically effective amount. A therapeutically effective amount is an amount effective to ameliorate or prevent the symptoms, or onset or progression of clinical disease of, the subject being treated. Determination of a therapeutically effective amount is well within the capabilities of those skilled in the art, especially in light of the detailed disclosure provided herein. For example, as described supra, in certain instances an effective amount of a compound of the invention may be defined by the ability of the compound to prevent a given amount of tau phosphorylation or accumulation.

For systemic administration, a therapeutically effective dose can be estimated initially from in vitro assays. For example, a dose can be formulated in animal models to achieve a circulating concentration range that includes the ICₛₒ as determined in cell culture. Such information can be used to more accurately determine useful doses in humans.

Initial dosages can also be estimated from in vivo data, e.g., animal models, using techniques that are well known in the art and the specific techniques described herein. One having ordinary skill in the art could readily optimize administration to humans based on animal data.

The amount of molecules administered will, of course, be dependent on the subject being treated, on the subject's weight, the severity of the affliction, the manner of administration and the judgment of the prescribing physician.

The therapy may be repeated intermittently while symptoms detectable or even when they are not detectable (e.g., to prevent to onset of symptoms). The therapy may be provided alone or in combination with other drugs. In some embodiments, an endothelin antagonist may be used in combination with an acetylcholinesterase inhibitor such as donepezil, rivastigmine, galantamine, vitamin E, or an anti-inflammatory drug such as a nonsteroidal anti-inflammatory drug (NSAID) (De La Garza, 2003). Non-limiting examples NSAIDs include, ibuprofen, ketoprofen, piroxicam, naproxen, naproxen sodium, sulindac, aspirin, choline subsalicylate, diflunisal, oxaprozin, diclofenac sodium delayed release, diclofenac potassium immediate release, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate.

Methods for estimating dose conversions between animal models and humans have previously been developed. In general these algorithms have been used to extrapolate an animal dose to a dose that would be tolerated by a human. For example, methods for dose conversion have previously been disclosed by Freireich et al. (1966). The conversion methods taught by Freireich calculate equivalent doses between species using surface area (m²) rather than mass (kg), a method that correlates much more closely to actual data than body mass conversions. Specifically, Freireich teaches how to use an animal 10% lethal dosage (LD₁₀) value to estimate the maximum tolerated doses in a human. Freireich also discussed method for converting a dose in mg/kg to a dose in mg/m² by using the “km” conversion factor for the given animal. For example, in the case of a laboratory mouse the km is approximately 3.0. Thus, in mice mg/m²=km (3.0 for mice)×dose in mg/kg.

More recent studies regarding species dose scaling have further elaborated upon the methods of Freireich. These newer studies have reduced error associated with conversion between species to determine human tolerable doses. For example, Watanabe et al. (1992) describes that a conversion of doses between species using body surface area may not be the most accurate method per se for predicting a human equivalent dosage. Nonetheless, the scaling factors set forth by Watanabe yield results that are with-in the margin of error of the older Freireich conversions. Currently accepted methods for determining a proper starting dose in humans expand upon the methods set forth by Freireich. For example, Mahmood et al. (2003) provides a discussion regarding the choice of a proper starting dose in humans given dose studies in animals.

B. Toxicity

Preferably, a therapeutically effective dose of an endothelin antagonist described herein will provide therapeutic benefit without causing substantial toxicity.

Toxicity of the molecules described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Proteins which exhibit high therapeutic indices are preferred. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975).

C. CNS Targeted Therapy

In some embodiments, an endothelin receptor antagonist may be CNS targeted. A variety of molecules are known to confer CNS targeting. For instance, certain antibodies are known to cross the BBB, thus such antibodies may be used to transport a payload, such as an endothelin receptor antagonist to the CNS. Some specific antibodies that may be used include but are not limited to antibodies to transferrin receptors (e.g., OX26) or antibodies to the insulin receptor (Schnyder & Huwyler, 2005). Other polypeptides may also be used to target the CNS such as cationized albumin. Thus, polypeptide CNS targeting agents may in some aspects, be bound to a endothelin receptor antagonist for use according to the invention. In some very specific cases, a peptide (or polypeptide) endothelin receptor antagonist may be provided as a fusion protein with a CNS targeting polypeptide. In still other cases nanoparticles such as Polysorbate 80-coated polybutylcyanoacrylate nanoparticles may be used to deliver compositions to the CNS (Olivier, 2005). In still further aspects, CNS targeting polypeptides may be conjugated to liposomes to form CNS targeting complexes (Schnyder & Huwyler, 2005). Furthermore, peptide and polypeptide endothelin receptor antagonist may be targeted to the CNS by glycosylation, for example as described in Egleton & Davis (2005). In yet further aspects, viral vectors may be used to targeted delivery of peptides or polypeptides to the CNS. For example, lentiviral vector systems for polypeptide delivery are known in the art, see for example Spencer & Verma (2007).

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Animals

Female athymic nude mice (NCI-nu) were purchased from the Animal Production Area of the National Cancer Institute—Frederick Cancer Research Facility (Frederick, Md.). The mice were housed and maintained in specific pathogen-free conditions in facilities approved by the American Association for Accreditation of Laboratory Animal Care and in accordance with all current regulations and standards of the US Department of Agriculture, the US Department of Health and Human Services, and the National Institutes of Health. The mice were used in these experiments in accordance with institutional guidelines when they were 8 to 12 weeks old.

Cell Culture and Reagents

Human breast cancer cell line MDA-MB-231 was maintained as monolayer cultures in a complete Eagle minimum essential medium (CMEM) supplemented with 10% fetal bovine serum (HyClone, Logan, Utah), L-glutamine pyruvate, nonessential amino acids, two-fold vitamin solution, and penicillin-streptomycin (GIBCO/Invitrogen, Carlsbad, Calif.). Murine astrocytes were isolated from neonatal mice homozygous for a temperature-sensitive SV40 large Tantigen (H-2K b-tsA58 mice; CBA/ca×C57BL/10 hybrid; Charles River Laboratories, Wilmington, Mass.) and established in culture in our laboratory as described in detail previously (Kim et al., 2011). To determine whether murine astrocytes can induce resistance All reagents used for tissue culture were free of endotoxin as determined by the limulus amebocyte lysate assay (Associate of Cape Cod, Woods Hole, Mass.), and the cell lines were free of the following murine pathogens: Mycoplasma spp, Hantan virus, hepatitis virus, minute virus , adenovirus (MAD1, MAD2), cytomegalovirus, ectromelia virus, lactate dehydrogenase-elevating virus, polyma virus, and Sendai virus (assayed by the Research Animal Diagnostic Laboratory, University of Missouri, Columbia, Mo.). Cells used in this study were from frozen stock, and all experiments were carried out within 10 in vitro passages after thawing.

IL-6, IL-8, VEGF, and ET-1 were purchased from R&D system (Minneapolis, Minn.). BQ123 (endothelin receptor A antagonist) and BQ788 (endothelin receptor B antagonist) were purchased from Sigma-Aldrich (St. Louis, Mo.). All of the chemicals were dissolved in dimethyl sulfoxide (DMSO), and all other reagents were of analytical reagent grade or better.

Orthotopic Implantation of Cancer Cells to Produce Brain Metastasis

To produce tumors, the human breast cancer, MDA-MB-231 cells were into the carotid of the mouse for the generation of brain metastases (Schackert and Fidler, 1988) and orthtopic injection to mammary fat pad. Cells were harvested from subconfluent cultures by a brief exposure to 0.25% trypsin and 0.02% EDTA, Trypsinization was stopped by replacing the trypsin-EDTA with medium containing 10% fetal bovine serum, and the cells were washed once in serum-free medium and resuspended in Ca²⁺-/Mg²⁺-free Hank's balanced salt solution. Cell viability was determined by trypan blue exclusion, and only single-cell suspensions of more than 95% viability were used for injection. Around 105 cells in 100 μL HBSS were injected the carotid artery and fat pad.

Collection of Gene Expression Data and Data Analysis

Total RNA was extracted from cancer tissues by using the mirVana miRNA Isolation Kit (Ambion, Tex.) according to the manufacturer's protocol. The integrity of the large RNA fraction was determined with an Experion Bioanalyzer (Bio-Rad, Calif., USA) as a surrogate for mRNA quality control. Biotin-labeled cRNA samples for hybridization were prepared by using Illumina Total Prep RNA Amplification Kit (Ambion). Each of 1.5 μg biotinylated cRNA was hybridized to T12-HumanHT-12 v4.0 Expression BeadChip. After the BeadChips were scanned with an Illumina BeadArray Reader, the microarray data were normalized using the quantile normalization method in the Linear Models for Microarray Data package in the R language environment (Bolstad et al., 2003). All statistical analysis was performed using BRB Arraytools Version 4.0. (Biometrics Research Branch, NCI, Bethesda, Md., Vol. 3.6). Cluster analysis was done with Cluster and Treeview (software manual). Microarray data was uploaded to Ingenuity Pathway Analysis (IPA Ingenuity System, Inc, Redwood City, Calif.) software, which was used for generation molecular and cellular functional analysis.

Real Time PCR

To determine whether ETs upregulation in astrocytes culture with tumor cells, we performed in vitro coculture experiments. Astrocytes and NIH 3T3 fibroblasts were transfected with green fluorescent protein (GFP) genes. The GFP-labeled astrocytes, GFP-labeled 3T3, and MDA-MB-231 cells were plated alone or as co-cultures at a cell ratio of 1:1 onto each of the sterile 6-well tissue culture multiwall dish. Total RNA was extracted from cells after 48 hr incubation. To determine ET expression in treatment of IL-6, IL-8, and VEFG, the astroctyes were treated with 5 ng/ml of IL-6, IL-8, or VEFG for 24 hrs. ET-1 (1 μM) was positive control. To determine endothelin antagonist effect on APOE and TAU mRNA expression, the astroctyes were preincubated for 2 hrs with 100 nM BQ123 or BQ788 alone or in combination, then the cells were further incubated with 1 μM ET-1 for 24 hrs. After treatment, total RNA was extracted from the collected cells using the Qiagen RNeasy mini kit (Qiagen, Valencia, Calif.) according to the manufacturer's instructions. First-strand cDNA was synthesized from 5 μg RNA using SuperScript III reverse transcriptase (Invitrogen, Carlsbad, Calif.). RT-PCR was performed using TaqMan® Universal PCR MasterMix and quantified with an ABI 7500 real time PCR system (Applied Biosystems, Foster City, Calif.). The TaqMan® gene expression assays used for murine ET-1, murine ET-2, murine ET-3, murine ETAR, murine ETBR, murine APOE, and murine TAU were Mm00438656-m1, Mm00432983-m1, Mm00432986-m1, Mn01243722_m1, Mn00432989_m1, Mm00437573_m1, and Mm00521988_m1 respectively (Applied Biosystems). 18S rRNA was used as an endogenous control. Relative mRNA expression was calculated according to the AACt method, and expressed as mean±S.D. of mRNA relative to that of control.

### Example 2

### Inhibition of Tau Production by Blockade of the Endothelin Receptor Axis

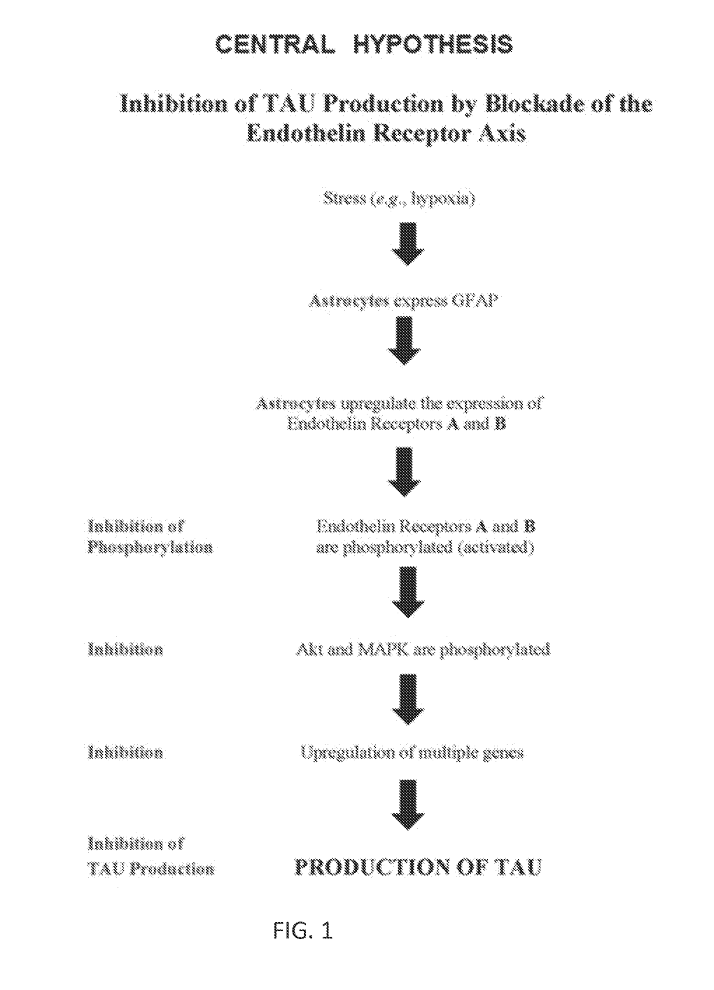

The major hypothesis that was tested deals with production of TAU by activated astrocytes (GFAP positive) which have established gap junction channels with adjacent cells e.g., endothelium, neurons, tumor cells. Without wishing to be bound by any theory, the hypothesis that was tested is outlined in FIG. 1.

Astrocytes can become activated and express GFAP in response to stressors such as: hypoxia, inflammation, VEGF, IL-6, and/or IL-8.

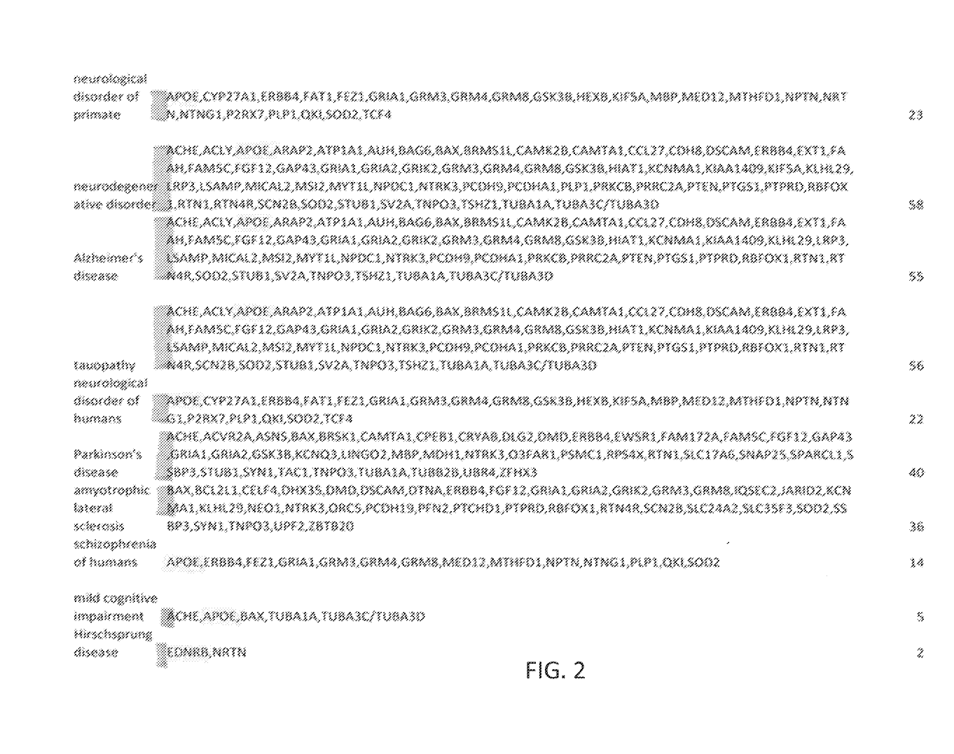

Altered expression of genes in astrocytes are shown in FIG. 2.

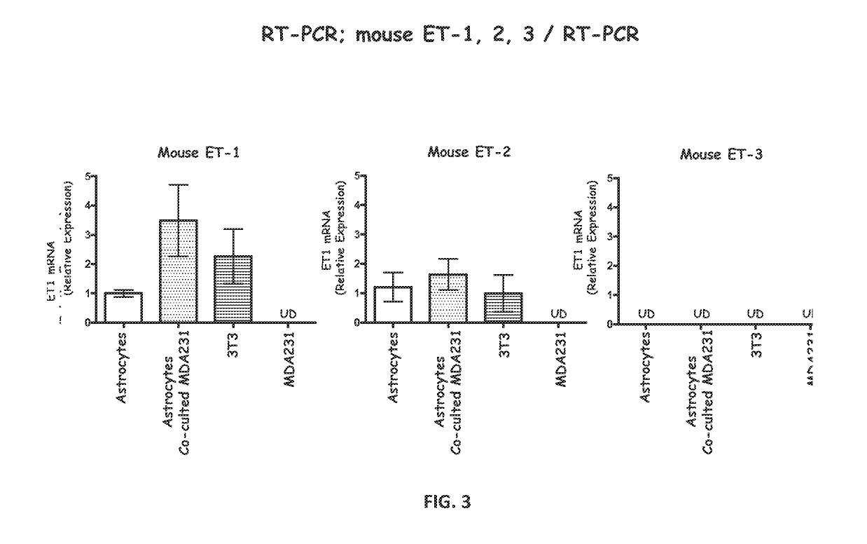

Next, the inventor determined what is the signal that astrocytes send to tumor cells to upregulate gene expression for these sets of genes. As a result of the RT-RCR tests described above, an increase in endothelin-1 was observed in astrocytes cultured in MDA231, as compared to control astrocytes (FIG. 3).

The data shows that ET1 produced by astrocytes binds to endothelin receptors A and B of tumor cells. Activation (phosphorylation) of the receptors leads to activation of Akt and MAPK pathways followed by upregulation of survival genes and chemoresistance. Inhibition of endothelin receptors phosphorylation presents the upregulation of survival genes.

Tau is a microtubule-associated protein with multiple phosphorylation sites. Hyper phosphorylation of Tau in Alzheimer's disease (AD) is promoted by several kinases. Inside neurons, Tau can form neuro fibrillary tangles leading to microtubule disintegration and collapse of the neuron transport system and later to death of the cells. APOE can alter Tau phosphorylation and, thus, potentially affect the accumulation of NFT (neurofibrillary tangles consisting of phosphorylated Tau protein)

Endothelial cells produce ET1 and ET2. Astrocyte's ET_{A}R, ET_{B}R are phosphorylated, and Tau is produced.

Tumor cells were shown to upregulate pAkt and pMAPK. Tumor cells can result in increased expression of genes (4000), and Tau is produced.

In certain pathology states in brain tissue, astrocytes are activated (GFAP). Then ETA and ETB are phosphorylated, and tau is produced. Tau is produced by astrocytes in response to the stress of hypoxia, trauma/inflammation, or cancer.

The results show that treatment of a tauopathy could be accomplished via inhibition of ET_{A}R and ET_{B}R phosphorylation, this can then reduce or prevent activation of pAkt/pMAPK, which can then reduce or prevent upregulation of genes, and result in inhibition of Tau.

Induction of Brain Hypoxia

A mouse was anesthetized with Nembutal (0.5 g/kg) and fixed in supine position with neck extended. Midline neck incision was made and common carotid artery was dissected upward to the bifurcation of internal and external carotid arteries. Common carotid artery was ligated at lower level with 6-0 black silk and internal carotid artery was canulated with 30 G-sized needle to inject cells in 50 ml of Ca²⁺-/Mg²⁺-free HBSS. Common carotid artery was ligated at the bifurcation level with 6-0 black silk and operative wound was closed with skin staples.

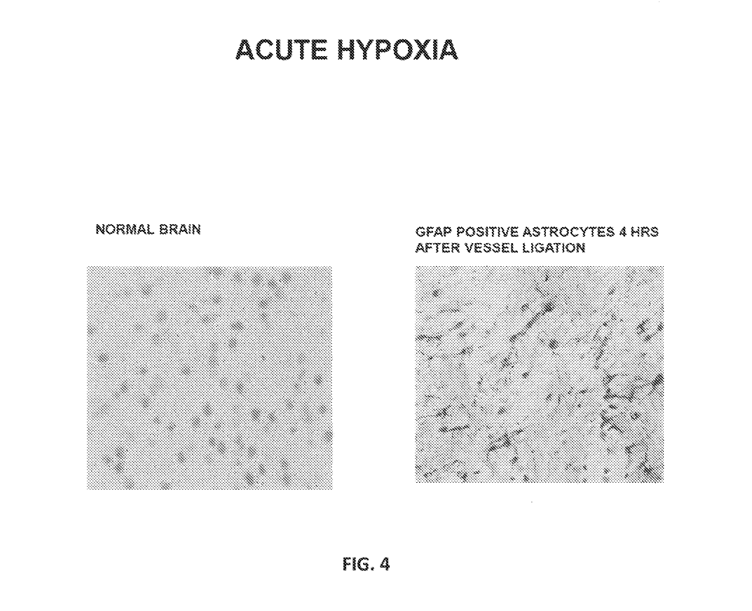

Astrocytes are GFAP-positive. Results are shown in FIG. 4.

Brain Parenchymal Wound

Brain parenchymal wound was created by inserting a needle (32 G-21 G) by stereotactic injection unit. In brief, a mouse was anesthetized with Nembutal (0.5 g/kg) and fixed in the stereotactic injection unit. Skin incision was made in the forehead and a hole was made in the calvaria with a 21 G needle. Injection needle was inserted into the brain parenchyma (4-mm depth) and withdrawn. Hole was blocked with bone wax and skin incision was closed with skin staples.

Brain was harvested after 1, 2, 4, and 5 days after creating brain parenchyma wound. Tissues were processed to make paraffin block (formalin-fixed) and stained for GFAP (DAB) or GFAP (green)/TAU (red) (immunofluorescent) staining.

Astrocytes were observed to be GFAP-positive and tau-positive near the wound site. Co-localization of GFAP and Tau was observed in astrocytes.

Real Time PCR

To determine whether ETs are upregulated in astrocytes cultured with tumor cells, we performed in vitro coculture experiments. Astrocytes and NIH 3T3 fibroblasts were transfected with green fluorescent protein (GFP) genes. The GFP-labeled astrocytes, GFP-labeled 3T3, and MDA-MB-231 cells were plated alone or as co-cultures at a cell ratio of 1:1 onto each sterile 6-well tissue culture multiwall dish. Total RNA was extracted from cells after 48 hr incubation. To determine ETs expression in treatment of IL-6, IL-8, and VEFG, the astroctyes were treated 5 ng/ml of IL-6, IL-8, or VEFG for 24 hrs. ET-1 (1 μM) as positive control. To determine endothelin antagonist effect on APOE and TAU mRNA expression, the astroctyes were preincubated for 2 hrs with 100 nM BQ123 or BQ788 alone or in combination, then the cells were further incubated with 1 μM ET-1 for 24 hrs. After treatment, total RNA was extracted from the collected cells using the Qiagen RNeasy mini kit (Qiagen, Valencia, Calif.) according to the manufacturer's instructions. First-strand cDNA was synthesized from 5 μg RNA using SuperScript III reverse transcriptase (Invitrogen, Carlsbad, Calif.). RT-PCR was performed using TaqMan® Universal PCR MasterMix and quantified with an ABI 7500 real time PCR system (Applied Biosystems, Foster City, Calif.). The TaqMan® gene expression assays used for murine ET-1, murine ET-2, murine ET-3, murine ETAR, murine ETBR, murine APOE, and murine TAU were Mm00438656-m1, Mm00432983-m1, Mm00432986-m1, Mn01243722m1, Mn00432989m1, Mm00437573_m1, and Mm00521988_m1 respectively (Applied Biosystems). 18S rRNA was used as an endogenous control. Relative mRNA expression was calculated according to the AACt method and expressed as mean±S.D. of mRNA relative to that of control.

Astrocytes Cultured Under Hypoxic Conditions

For incubations in hypoxia, the astrocytes were placed in a chamber that was flushed with a gas mixture of 5% CO2/95% N2. Oxygen concentrations within the chamber were maintained for 24 hr at 0.5% using a ProOx 110 oxygen regulator (Biospherix) for indicated time period.

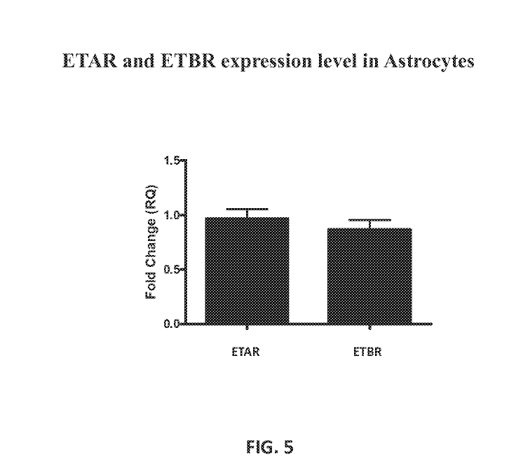

ETAR and ETBR expression level in Astrocytes are shown in FIG. 5.

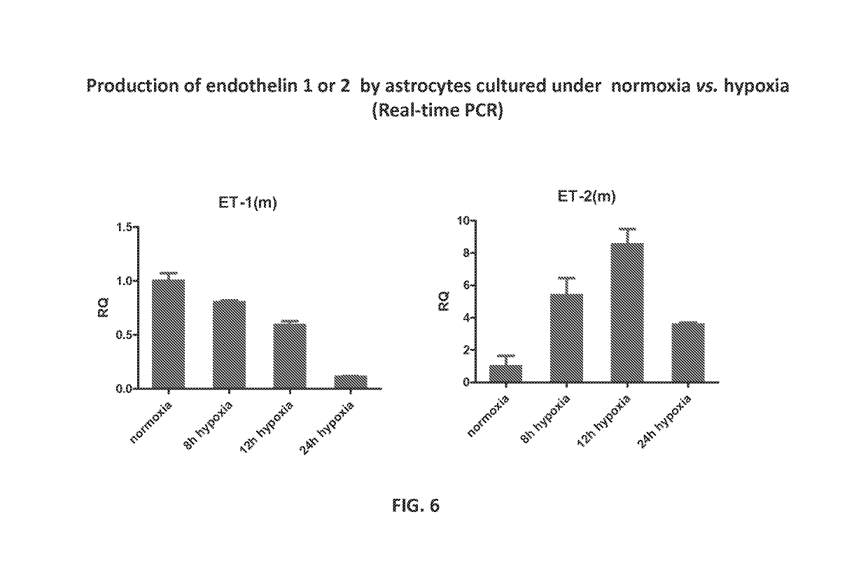

Production of endothelin 1 or 2 by astrocytes cultured under normoxia vs. hypoxia (Real-time PCR) results are shown in FIG. 6.

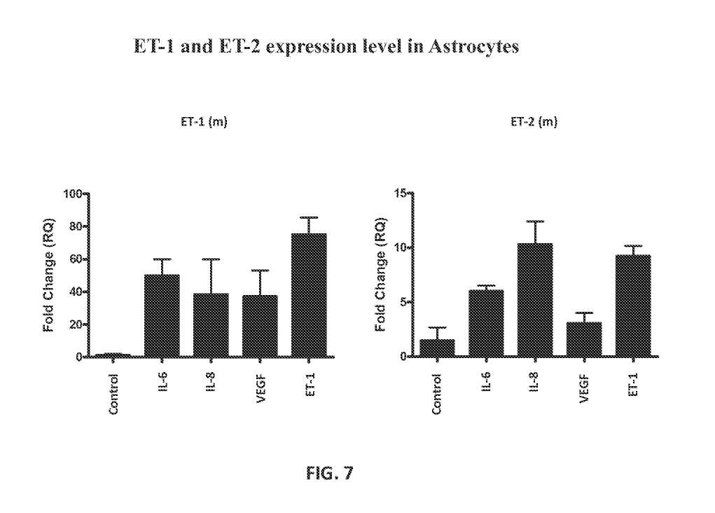

ET-1 and ET-2 expression level in Astrocytes is shown in FIG. 7.

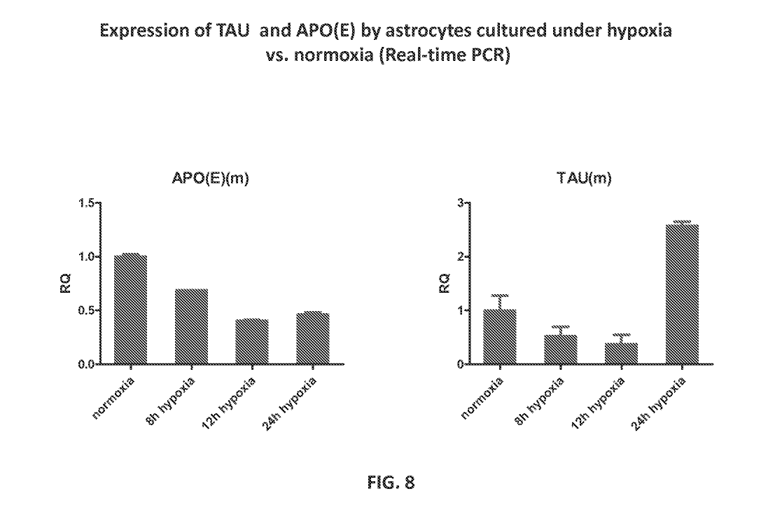

Expression of TAU and APO(E) by astrocytes cultured under hypoxia vs. normoxia (Real-time PCR) results are shown in FIG. 8.

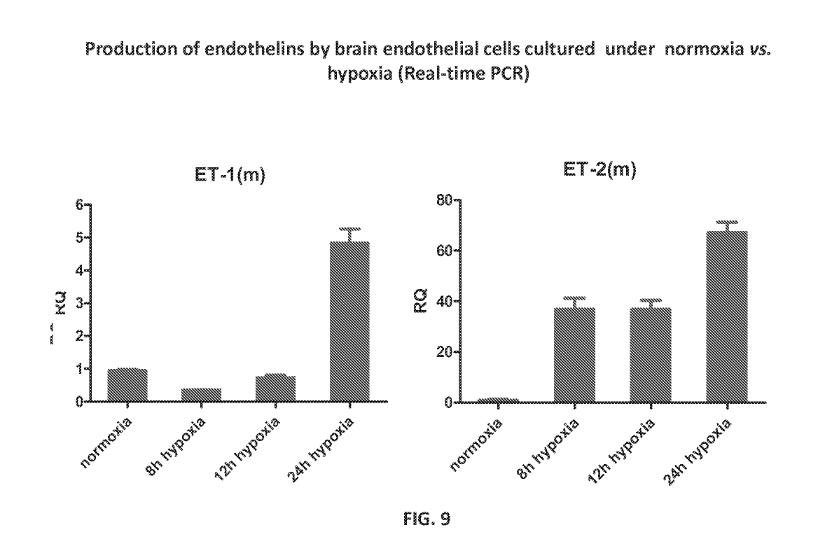

Hypoxia effects on astrocytes are elevated tau (pathology) and decreased APOE (pathology). Production of endothelins by brain endothelial cells cultured under normoxia vs. hypoxia (Real-time PCR) results are shown in FIG. 9.

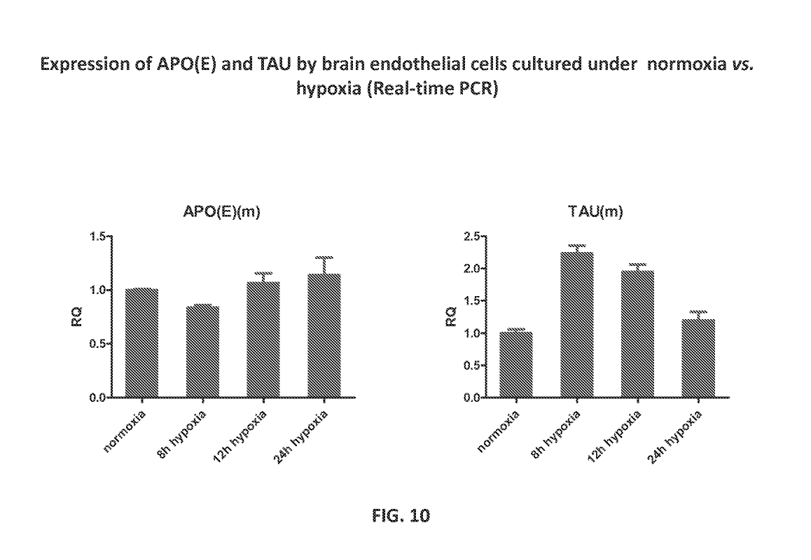

Expression of APO(E) and TAU by brain endothelial cells cultured under normoxia vs. hypoxia (Real-time PCR) results are shown in FIG. 10.

### EXAMPLE 3

### Treatment of Tauopathy with Dual Endothelin Receptor Inhibitor, Such as BQ123 Plus BQ788 or PD145065

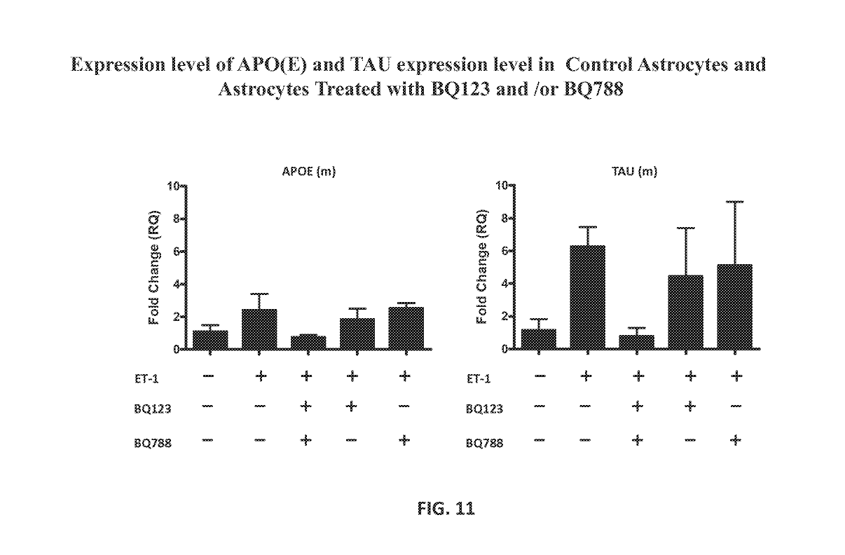

Expression level of APO(E) and Tau expression level in control astrocytes and astrocytes treated with BQ123 and/or BQ788 or PD145065 results are shown in FIG. 11.

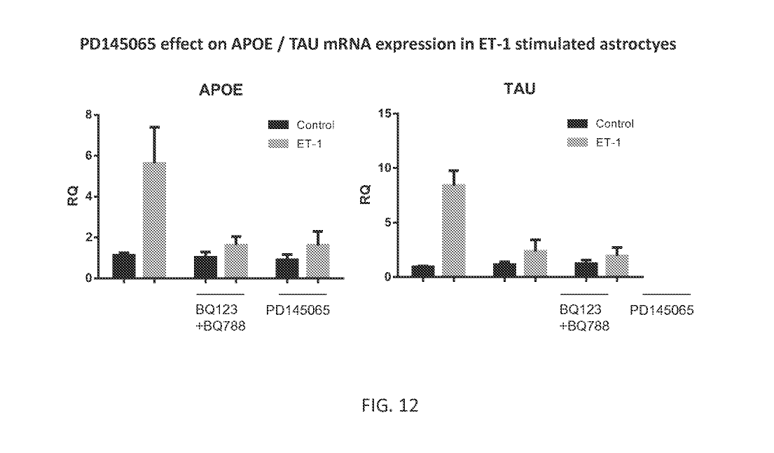

PD145065 was observed to have an effect on APOE/TAU mRNA expression in ET-1 stimulated astroctyes as shown in FIG. 12.

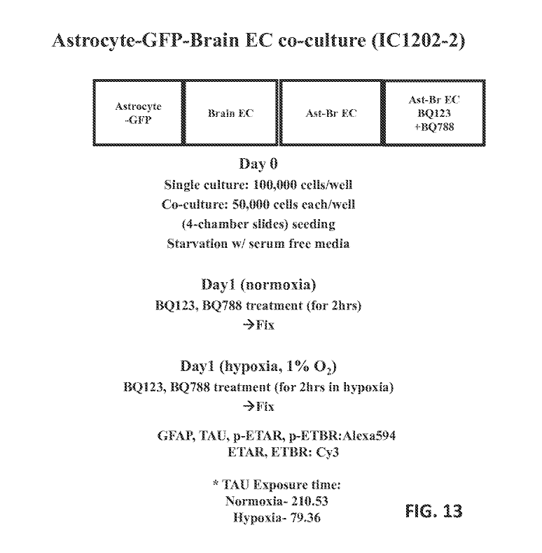

Astrocyte-GFP-Brain EC co-culture methods are shown in FIG. 13. Tau expression under normal and hypoxic conditions is shown in FIG. 13, and an increase in tau expression was observed under hypoxic conditions.

Inhibition of ETAR and ETBR phosphorylation reduces tau to background level. This is a positive finding because Tau is an essential microtubule-associated protein. Pathology is associated with over production of Tau and its hyperphosphorylation when Tau begins to form NFT (neurofibrillary tangles) inside nerve cell bodies.

Inhibition of Gene Expression in Tumor Cells by Inhibition of Endothelin Receptor A and B Phosphorylation

MDA231 cells were cultured as a single or co-cultured with mouse astrocytes (ratio 1:1) with or without treatment with 1 mM of BQ123 and BQ788 for 48 hrs. Gene array and class comparison of genes were done as described. Genes up-regulated with co-culture of MDA231 and genes down-regulated by the treatment with BQ123 and BQ788 were identified. Genes down-regulated by the treatment with BQ123 and BQ788 were further analyzed as Alzheimer's disease-, APOE- or TAU-related genes by literature search.

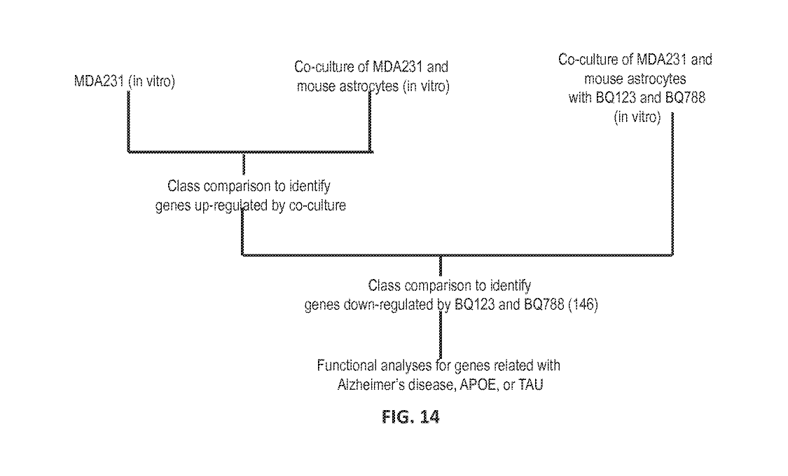

Methodology to identify genes whose expression is altered by BQ123 and BQ788 is shown in FIG. 14. The following genes were found to be directly related genes (double underline) or indirectly related genes (single underline):

| Alzheimer's Disease | TAU | APO(E) | |
|---|---|---|---|
| ABCF3 | ABCF3 | ABCF3 | ATP-binding cassette, sub-family |
| | | | F (GCN20), member 3 |
| ACOT7 | ACOT7 | ACOT7 | acyl-CoA thioesterase 7 |
| ACPT | ACPT | ACPT | acid phosphatase, testicular |
| ACTG2 | ACTG2 | ACTG2 | actin, gamma 2, smooth muscle, |
| | | | enteric |
| ACTN4 | ACTN4 | ACTN4 | actinin, alpha 4 |
| ADM | ADM | ADM | adrenomedullin |
| ANKRD11 | ANKRD11 | ANKRD11 | ankyrin repeat domain 11 |
| ARHGAP22 | ARHGAP22 | ARHGAP22 | Rho GTPase activating protein 22 |
| ART3 | ART3 | ART3 | ADP-ribosyltransferase 3 |
| CCDC19 | CCDC19 | CCDC19 | coiled-coil domain containing 19 |
| CCDC94 | CCDC94 | CCDC94 | coiled-coil domain containing 94 |
| CCL2 | CCL2 | CCL2 | chemokine (C-C motif) ligand 2 |
| CDK5RAP1 | CDK5RAP1 | CDK5RAP1 | CDK5 regulatory subunit |
| | | | associated protein 1 |
| CENPE | CENPE | CENPE | centromere protein E, 312 kDa |
| CENPF | CENPF | CENPF | centromere protein F, |
| | | | 350/400 kDa (mitosin) |
| CHERP | CHERP | CHERP | calcium homeostasis endoplasmic |
| | | | reticulum protein |
| CHMP6 | CHMP6 | CHMP6 | charged multivesicular body |
| | | | protein 6 |
| CIR1 | CIR1 | CIR1 | corepressor interacting with |
| | | | RBPJ, 1 |
| COL13A1 | COL13A1 | COL13A1 | collagen, type XIII, alpha 1 |
| COL18A1 | COL18A1 | COL18A1 | collagen, type XVIII, alpha 1 |
| COL1A1 | COL1A1 | COL1A1 | collagen, type I, alpha 1 |
| COL4A1 | COL4A1 | COL4A1 | collagen, type IV, alpha 1 |
| COL5A1 | COL5A1 | COL5A1 | collagen, type V, alpha 1 |
| COL5A2 | COL5A2 | COL5A2 | collagen, type V, alpha 2 |
| COPG | COPG | COPG | coatomer protein complex, |
| | | | subunit gamma |
| CPA4 | CPA4 | CPA4 | carboxypeptidase A4 |
| CTGF | CTGF | CTGF | connective tissue growth factor |
| CTSH | CTSH | CTSH | cathepsin H |
| DGCR6 | DGCR6 | DGCR6 | DiGeorge syndrome critical region |
| | | | gene 6 |
| DKK3 | DKK3 | DKK3 | dickkopf homolog 3 (Xenopus |
| | | | laevis) |
| DNM1 | DNM1 | DNM1 | dynamin 1 |
| DNMT1 | DNMT1 | DNMT1 | DNA (cytosine-5-)- |
| | | | methyltransferase 1 |
| DUSP1 | DUSP1 | DUSP1 | dual specificity phosphatase 1 |
| EDN1 | EDN1 | EDN1 | endothelin 1 |
| EED | EED | EED | embryonic ectoderm development |
| EGR1 | EGR1 | EGR1 | early growth response 1 |
| EIF5B | EIF5B | EIF5B | eukaryotic translation initiation |
| | | | factor 5B |
| EPM2AIP1 | EPM2AIP1 | EPM2AIP1 | EPM2A (laforin) interacting |
| | | | protein 1 |
| ERC1 | ERC1 | ERC1 | ELKS/RAB6-interacting/CAST |
| | | | family member 1 |
| FOS | FOS | FOS | FBJ murine osteosarcoma viral |
| | | | oncogene homolog |
| FRMD4A | FRMD4A | FRMD4A | FERM domain containing 4A |
| FSTL1 | FSTL1 | FSTL1 | follistatin-like 1 |
| GPRC5A | GPRC5A | GPRC5A | G protein-coupled receptor, family |
| | | | C, group 5, member A |
| HDLBP | HDLBP | HDLBP | high density lipoprotein binding |
| | | | protein |
| HLA-E | HLA-E | HLA-E | major histocompatibility complex, |
| | | | class I, E |
| HNRPA1L3 | HNRPA1L3 | HNRPA1L3 | heterogeneous nuclear |
| | | | ribonucleoprotein A1 pseudogene 7 |
| HSD3B7 | HSD3B7 | HSD3B7 | hydroxy-delta-5-steroid |
| | | | dehydrogenase, 3 beta- and |
| | | | steroid delta-isomerase 7 |
| HSPA5 | HSPA5 | HSPA5 | heat shock 70 kDa protein 5 |
| | | | (glucose-regulated protein, |
| | | | 78 kDa) |
| IFIT3 | IFIT3 | IFIT3 | interferon-induced protein with |
| | | | tetratricopeptide repeats 3 |
| IL23A | IL23A | IL23A | interleukin 23, alpha subunit p19 |
| IL7R | IL7R | IL7R | interleukin 7 receptor |
| KIAA1324 | KIAA1324 | KIAA1324 | KIAA1324 |
| KRT17 | KRT17 | KRT17 | keratin 17 |
| LAMA5 | LAMA5 | LAMA5 | laminin, alpha 5 |
| LAMC2 | LAMC2 | LAMC2 | laminin, gamma 2 |
| LEO1 | LEO1 | LEO1 | Leo1, Paf1/RNA polymerase II |
| | | | complex |
| | | | component, homolog (S. |
| | | | cerevisiae) |
| LIMA1 | LIMA1 | LIMA1 | LIM domain and actin binding 1 |
| LOC100128295 | LOC100128295 | LOC100128295 | similar to hCG1639947 |
| LOC100131688 | LOC100131688 | LOC100131688 | vomeronasal 1 receptor 68 |
| | | | pseudogene |
| LRR1 | LRR1 | LRR1 | leucine rich repeat protein 1 |
| LTBP3 | LTBP3 | LTBP3 | latent transforming growth factor |
| | | | beta binding |
| | | | protein 3 |
| LUC7L3 | LUC7L3 | LUC7L3 | LUC7-like 3 (S. cerevisiae) |
| MAMDC2 | MAMDC2 | MAMDC2 | MAM domain containing 2 |
| MED11 (includes | MED11 | MED11 | mediator complex subunit 11 |
| EG: 1001485041) | (includes | (includes | |
| | EG: 1001485041) | EG: 1001485041) | |
| mir-939 | mir-939 | mir-939 | microRNA 939 |
| MKI67 | MKI67 | MKI67 | antigen identified by monoclonal |
| | | | antibody Ki-67 |
| MRTO4 | MRTO4 | MRTO4 | mRNA turnover 4 homolog (S. |
| | | | cerevisiae) |
| MSI2 | MSI2 | MSI2 | musashi homolog 2 (Drosophila) |
| MYH10 | MYH10 | MYH10 | myosin, heavy chain 10, non- |
| | | | muscle |
| MYH9 | MYH9 | MYH9 | myosin, heavy chain 9, non- |
| | | | muscle |
| MYH9 | MYH9 | MYH9 | myosin, heavy chain 9, non- |
| | | | muscle |
| MYO5A | MYO5A | MYO5A | myosin VA (heavy chain 12, |
| | | | myoxin) |
| MYOF | MYOF | MYOF | myoferlin |
| NBPF11 (includes | NBPF11 | NBPF11 | neuroblastoma breakpoint family, |
| others) | (includes | (includes | member 11 |
| | others) | others) | |
| NBPF11 (includes | NBPF11 | NBPF11 | neuroblastoma breakpoint family, |
| others) | (includes | (includes | member 11 |
| | others) | others) | |
| NBPF11 (includes | NBPF11 | NBPF11 | neuroblastoma breakpoint family, |
| others) | (includes | (includes | member 11 |
| | others) | others) | |
| NCL | NCL | NCL | nucleolin |
| NCL | NCL | NCL | nucleolin |
| NCOR2 | NCOR2 | NCOR2 | nuclear receptor corepressor 2 |
| NEK11 | NEK11 | NEK11 | NIMA (never in mitosis gene a)- |
| | | | related kinase 11 |
| NOP2 | NOP2 | NOP2 | NOP2 nucleolar protein homolog |
| | | | (yeast) |
| NOP56 | NOP56 | NOP56 | NOP56 ribonucleoprotein |
| | | | homolog (yeast) |
| NPAS2 | NPAS2 | NPAS2 | neuronal PAS domain protein 2 |
| NPIP (includes others) | NPIP (includes | NPIP (includes | nuclear pore complex interacting |
| | others) | others) | protein |
| NRG1 (includes | NRG1 (includes | NRG1 (includes | neuregulin 1 |
| EG: 112400) | EG: 112400) | EG: 112400) | |
| NSRP1 | NSRP1 | NSRP1 | nuclear speckle splicing |
| | | | regulatory protein 1 |
| NSUN5 | NSUN5 | NSUN5 | NOP2/Sun domain family, |
| | | | member 5 |
| NUMA1 | NUMA1 | NUMA1 | nuclear mitotic apparatus protein 1 |
| OLFML3 | OLFML3 | OLFML3 | olfactomedin-like 3 |
| PDCD11 | PDCD11 | PDCD11 | programmed cell death 11 |
| PDLIM5 | PDLIM5 | PDLIM5 | PDZ and LIM domain 5 |
| PHACTR4 | PHACTR4 | PHACTR4 | phosphatase and actin regulator 4 |
| PHRF1 | PHRF1 | PHRF1 | PHD and ring finger domains 1 |
| PHYHD1 | PHYHD1 | PHYHD1 | phytanoyl-CoA dioxygenase |
| | | | domain containing 1 |
| PKP3 | PKP3 | PKP3 | plakophilin 3 |
| PNISR | PNISR | PNISR | PNN-interacting serine/arginine- |
| | | | rich protein |
| PNISR | PNISR | PNISR | PNN-interacting serine/arginine- |
| | | | rich protein |
| POLR3D | POLR3D | POLR3D | polymerase (RNA) III (DNA |
| | | | directed) polypeptide D, 44 kDa |
| PPP2CB | PPP2CB | PPP2CB | protein phosphatase 2, catalytic |
| | | | subunit, beta isozyme |
| PPP2R5D | PPP2R5D | PPP2R5D | protein phosphatase 2, regulatory |
| | | | subunit B′, delta |
| PRRC2C | PRRC2C | PRRC2C | proline-rich coiled-coil 2C |
| RAMP1 | RAMP1 | RAMP1 | receptor (G protein-coupled) |
| | | | activity modifying protein 1 |
| RANGRF | RANGRF | RANGRF | RAN guanine nucleotide release |
| | | | factor |
| RASSF1 | RASSF1 | RASSF1 | Ras association (RalGDS/AF-6) |
| | | | domain family member 1 |
| RASSF1 | RASSF1 | RASSF1 | Ras association (RalGDS/AF-6) |
| | | | domain family member 1 |
| RERE | RERE | RERE | arginine-glutamic acid dipeptide |
| | | | (RE) repeats |
| RERE | RERE | RERE | arginine-glutamic acid dipeptide |
| | | | (RE) repeats |
| RFC1 | RFC1 | RFC1 | replication factor C (activator 1) 1, |
| | | | 145 kDa |
| RN5S9 | RN5S9 | RN5S9 | RNA, 5S ribosomal 9 |
| RPL7L1 | RPL7L1 | RPL7L1 | ribosomal protein L7-like 1 |
| RPLP1 | RPLP1 | RPLP1 | ribosomal protein, large, P1 |
| RPS4X | RPS4X | RPS4X | ribosomal protein S4, X-linked |
| RRBP1 | RRBP1 | RRBP1 | ribosome binding protein 1 |
| | | | homolog g180 kDa (dog) |
| RRBP1 | RRBP1 | RRBP1 | ribosome binding protein 1 |
| | | | homolog 180 kDa (dog) |
| RRBP1 | RRBP1 | RRBP1 | ribosome binding protein 1 |
| | | | homolog 180 kDa (dog) |
| RUSC2 | RUSC2 | RUSC2 | RUN and SH3 domain containing 2 |
| SAA1 | SAA1 | SAA1 | serum amyloid A1 |
| SDCCAG8 | SDCCAG8 | SDCCAG8 | serologically defined colon cancer |
| | | | antigen 8 |
| SIGIRR | SIGIRR | SIGIRR | single immunoglobulin and toll- |
| | | | interleukin 1 receptor (TIR) |
| | | | domain |
| SLTM | SLTM | SLTM | SAFB-like, transcription modulator |
| SMG9 | SMG9 | SMG9 | smg-9 homolog, nonsense |
| | | | mediated mRNA decay factor (C. |
| | | | elegans) |
| SNORD3A | SNORD3A | SNORD3A | small nucleolar RNA, C/D box 3A |
| SPEN | SPEN | SPEN | spen homolog, transcriptional |
| | | | regulator (Drosophila) |
| SPP1 (includes | SPP1 (includes | SPP1 (includes | secreted phosphoprotein 1 |
| EG: 20750) | EG: 20750) | EG: 20750) | |
| SPP1 (includes | SPP1 (includes | SPP1 (includes | secreted phosphoprotein 1 |
| EG: 20750) | EG: 20750) | EG: 20750) | |
| SRR | SRR | SRR | serine racemase |
| SRRM1 | SRRM1 | SRRM1 | serine/arginine repetitive matrix 1 |
| SRRM2 | SRRM2 | SRRM2 | serine/arginine repetitive matrix 2 |
| SRSF11 | SRSF11 | SRSF11 | serine/arginine-rich splicing factor 11 |
| STK11IP | STK11IP | STK11IP | serine/threonine kinase 11 |
| | | | interacting protein |
| SUSD2 | SUSD2 | SUSD2 | sushi domain containing 2 |
| TAGLN | TAGLN | TAGLN | transgelin |
| TGFB2 | TGFB2 | TGFB2 | transforming growth factor, beta 2 |
| THBS1 | THBS1 | THBS1 | thrombospondin 1 |
| TLN1 | TLN1 | TLN1 | talin 1 |
| TMEM171 | TMEM171 | TMEM171 | transmembrane protein 171 |
| TMEM204 | TMEM204 | TMEM204 | transmembrane protein 204 |
| TNC | TNC | TNC | tenascin C |
| TOM1L1 | TOM1L1 | TOM1L1 | target of myb1 (chicken)-like 1 |
| TRAF7 | TRAF7 | TRAF7 | TNF receptor-associated factor 7 |
| TRIOBP | TRIOBP | TRIOBP | TRIO and F-actin binding protein |
| TSPAN4 | TSPAN4 | TSPAN4 | tetraspanin 4 |
| U2AF2 | U2AF2 | U2AF2 | U2 small nuclear RNA auxiliary |
| | | | factor 2 |
| VGF | VGF | VGF | VGF nerve growth factor inducible |
| WDR73 | WDR73 | WDR73 | WD repeat domain 73 |
| ZEB1 | ZEB1 | ZEB1 | zinc finger E-box binding |
| | | | homeobox 1 |
| ZSWIM1 | ZSWIM1 | ZSWIM1 | zinc finger, SWIM-type containing 1 |

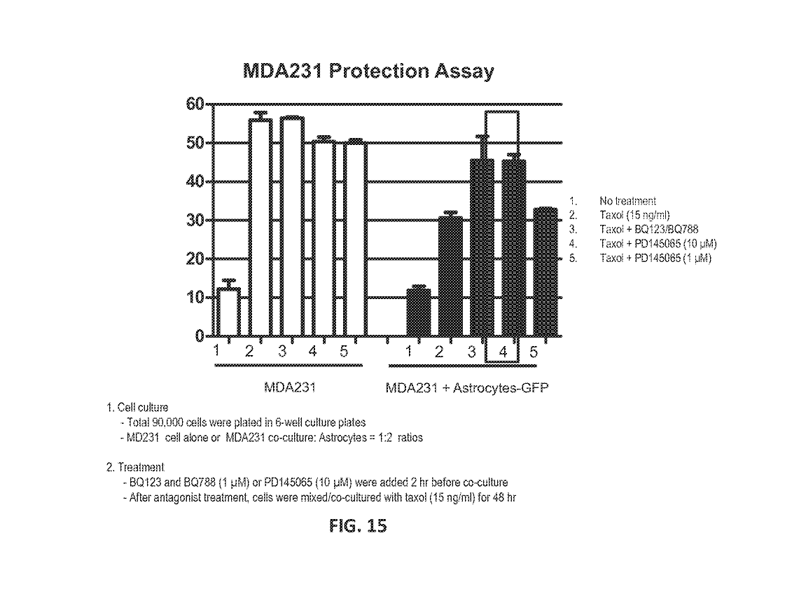

Next the inventor used the compound PD 145065, which is a dual endothelin receptor inhibitor (antagonist), in further experiments. The tumor cell -astrocyte protection assay (used as an example) showed that PD 145065 is as effective as the combination of BQ123 and BQ788 in blocking the ability of astrocytes protection of tumor cells from chemotherapy. The immunohistochemistry shows that PD145065 can prevent phosphorylation of both ETA and ETB receptors. Results are shown in FIG. 15. PD145065 may be administered orally to mice. Based on the in vitro data, it is anticipated that PD145065 may be used to decrease TAU in the brain in vivo.

### Example 4

### Treatment of Brain Trauma with a Dual Endothelin Receptor Inhibitor In Vivo

Wound Experiment

A mouse model of brain trauma was used to evaluate the effect of a dual endothelin receptor antagonist (PD 145065) on TAU expression after a brain trauma. A parenchyma mouse model of brain trauma was used such that mice received a stereotaxically administered wound to the brain Immunochemistry was performed on brain samples to evaluate GFAP and TAU expression.

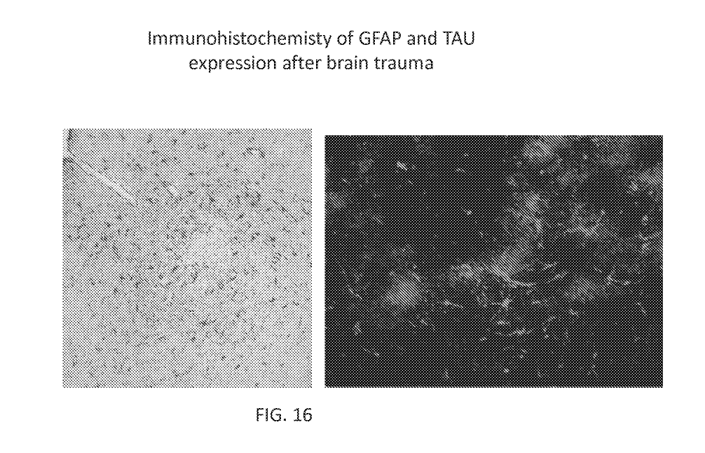

The mice were euthanatized 1, 2, or 4 days after creating the wound in the brain parenchyma. The brains were removed, fixed in formalin and stained for GFAP or (GFAP and TAU) using immunofluorescent staining. Results are shown in FIG. 16. Astrocytes were observed to express GFAP and TAU.

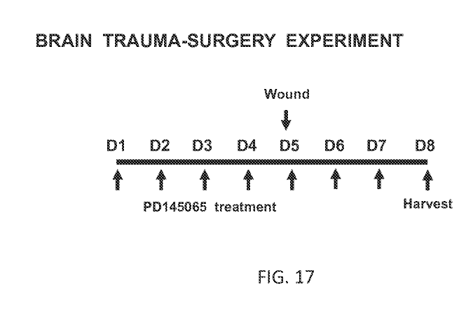

FIG. 17 shows an overview of the brain trauma surgery experiment. Mice were pretreated with PD145065 10 mg/kg/day i.p. injection for 5 days prior to brain wounding. Control mice received saline injections. Treatment continued for 3 days after wounding. The brains were harvested 3 days after wounding and immunofluorescent staining immunohistochemistry experiments were performed.

Results: TAU positive cells were observed in the brains of control mice but not in brains of mice pretreated in PD145065. Treatment with PD145065 also inhibited phosphorylation of endothelin receptors A and B. GFAP was observed to be unchanged in control and PD145065-treated mice, whereas TAU expression was observed to be significantly diminished or eliminated in the brains of PD145065-treated mice.

The wounds in brains of control mice were observed to contain cells that express phosphorylated ET_{A}R and ET_{B}R. In contrast, in the brains of mice pretreated with PD145065, the receptors were observed to have reduced phosphorylation or be not phosphorylated. In the brains of control mice and PD145065, pretreated mice contain activated (GFAP-positive) astrocytes. Astrocytes in control mice were observed to express TAU, whereas in PD145065 pretreated mice, TAU is negative.

These results demonstrate that a dual endothelin receptor antagonist may be used in vivo to reduce TAU expression in the brain after a brain trauma or wound.

### Example 5

### Treatment of Hypoxia with a Dual Endothelin Receptor Inhibitor In Vivo

Methods for Induction of Acute Brain Hypoxia

A mouse was anesthetized with Nembutal (0.5 g/kg) and fixed in supine position with neck extended. Midline neck incision was made and common carotid artery was dissected upward to the bifurcation of internal and external carotid arteries. Common carotid artery was ligated at lower level with 6-0 black silk, and internal carotid artery was canulated with 30 G-sized needle to inject cells in 50 ml of Ca++/Mg++-free HBSS. Common carotid artery was ligated at the bifurcation level with 6-0 black silk and operative wound was closed with skin staples.

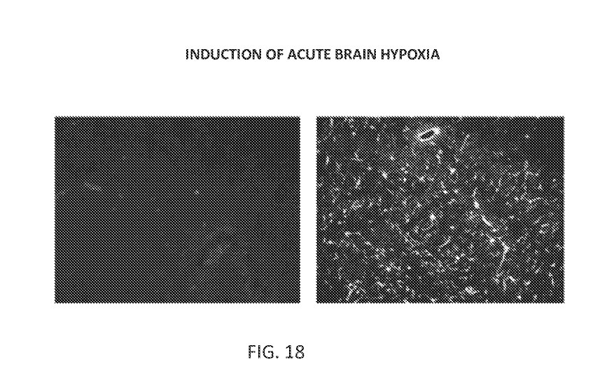

After generation of induction of acute brain hypoxia in mice, the brains were collected and evaluated using immunohistochemistry. Results are shown in FIG. 18. Astrocytes were observed to be GFAP-positive. Increased expression of TAU was observed in astrocytes from brains that were exposed to the acute brain hypoxia, as compared to control brain tissues.

Hypoxia Chamber

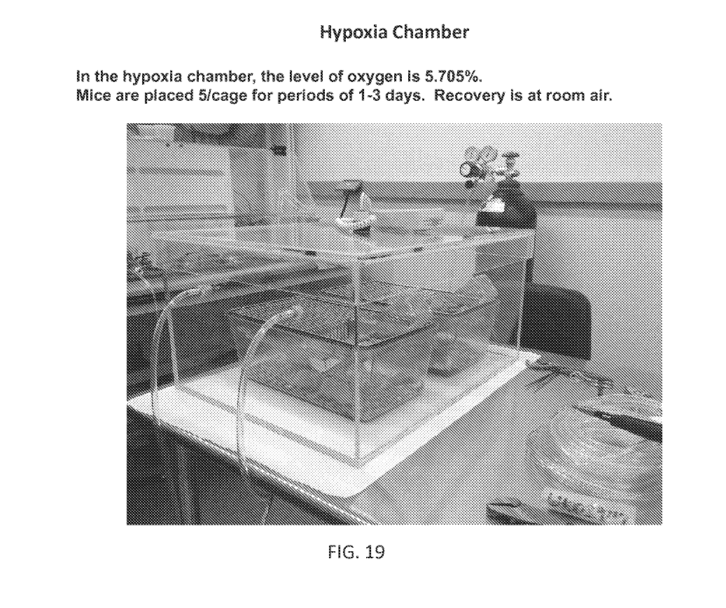

Mice were placed in a sealed plexiglass chamber with controlled input of oxygen-nitrogen gas to establish an oxygen level of 5.705% oxygen. A photo of the hypoxia chamber is shown in FIG. 19. One to 3 days later, the mice are euthanized or placed in room air (normoxia). The brains are harvested, fixed in formalin, or frozen for histology/immunohistochemistry examination. Staining for GFAP (astrocytes), TAU, endothelin receptor A or B (phosphorylation) was carried out.

Chronic Hypoxia Experiments with Dual Endothelin Receptor Inhibitor

Mice were placed in a hypoxia chamber for 1-3 days are taken out and divided into 2 groups: (1) treated with PD145065 (dual endothelin receptor inhibitor), or (2) saline. Treatments are by daily i.p. injections. The brains of control and treated mice are harvested 2-5 days later and processed for histological examination Immunohistochemistry for GFAP, TAU, ET_{A}R, and ET_{B}R (phosphorylation) was carried out.

TAU expression was observed in brains in vivo, using a mouse model of hypoxia. The following methods were used for the chronic hypoxia (hypoxia chamber) experiments. Mice were placed in a sealed plexiglass chamber with controlled input of oxygen-nitrogen gas to establish an oxygen level of 5.705% oxygen. One to 3 days later, the mice were euthanized or placed in room air (normoxia). The brains were harvested, fixed in formalin, or frozen for histology/immunohistochemistry examination. Staining for GFAP (astrocytes), TAU, endothelin receptor A or B phosphorylation was carried out. High levels of TAU expression was observed in hypoxic brains even after 120 hours recovery.

Administration of PD145065 inhibited TAU expression resulting from hypoxia, as shown with in vivo experiments. The following methods were used for testing the effects of PD145065 on chronic hypoxia. Mice were placed in a hypoxia chamber for 1-3 days. The mice were then removed from the hypoxia chambers and divided into 2 groups: (1) mice treated with PD145065 (dual endothelin receptor inhibitor); or (2) mice administered saline. Treatments are by daily i.p. injections. The mice remained under normoxic conditions (i.e., the mice were kept at room atmosphere), and then the brains of control and treated mice were harvested 2-5 days later and processed for histological examination. Immunohistochemistry for GFAP, TAU, ET_{A}R, and ET_{B}R phosphorylation was carried out. TAU expression levels in mice pretreated with PD145065 were significantly lower than in the brains of control mice.

Pretreatment with PD145065 was performed by the following method. TAU staining in brains of mice pretreated with PD145065 for 7 days prior to the induction of hypoxia for 48 hours. Following hypoxia, the mice were placed in normoxia.

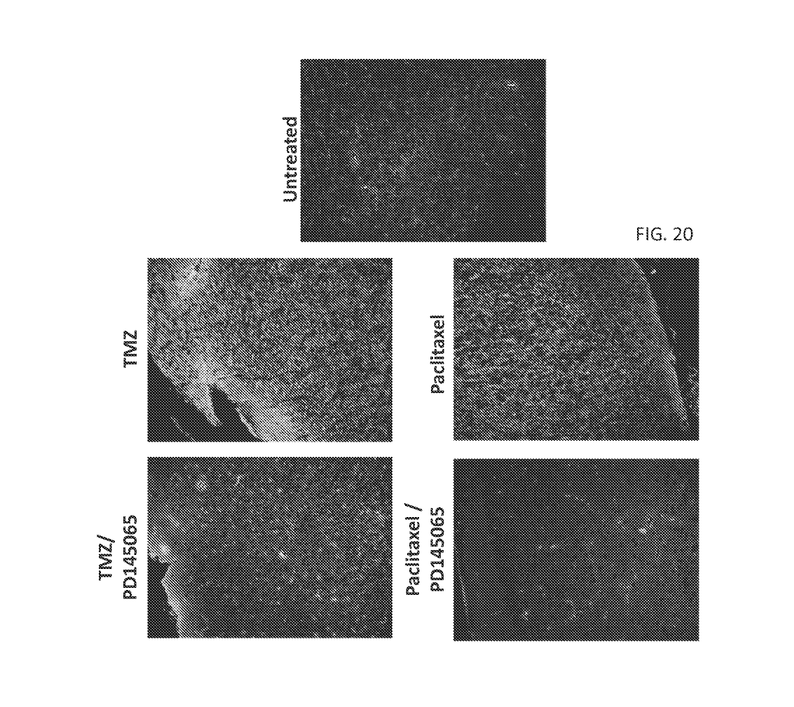

As shown in FIG. 20, pretreatment with PD145065 reduced tau expression in mice exposed to hypoxia or a brain trauma. These results demonstrate that a dual receptor inhibitor can reduce tau expression associated with brain hypoxia in vivo.

### Example 6

### Treatment of Chemo-Brain and Weight Loss Associated with Chemotherapeutic Administration with a Dual Endothelin Receptor Inhibitor In Vivo

The following methods were used to test for the in vivo effect of a dual endothelin receptor on a mouse model of chemo-brain. PD145065: SIGMA (SCP0143, 5 mg) was reconstituted in PBS containing 0.1% BSA. Temozolomide (TMZ): SIGMA (T2577-100 MG), was reconstituted in DMSO (10 mg/mL) with sonication and diluted in HBSS before treatment TAXOL (paclitaxel): Hospira (NDC 61703-342-09, 6 mg/mL) was diluted in saline before treatment. Mice were treated for 6 weeks with either TMZ (7.5 mg/kg, p.o., daily) or paclitaxel (8 mg/kg, i.p., twice per week) with or without PD145065 (10 mg/kg, i.p., daily). Treatment groups were as follows:

1. Normal control;

2. TMZ (oral administration of 7.5 mg/kg, daily);

3. TMZ+PD145065 (intra-peritoneal injection of 10 mg/kg, daily);

4. Paclitaxel (intra-peritoneal injection of 8 mg/kg, twice per week);

5. Paclitaxel+PD145065 (intra-peritoneal injection of 10 mg/kg, daily).

Mice were euthanized and the brains were collected and frozen (OCT) or placed in formalin and embedded in paraffin and then processed for IHC. Female nude mice were treated with Taxol (8 mg/kg) i.p. twice per week for six weeks. Mice exhibiting weight loss, dehydration, poor ambulation, and/or lethargy were euthanatized and their brains were frozen, sectioned and stained for TAU. Female nude mice were treated orally with 7.5 mg/kg TMZ every day for six weeks. Mice exhibiting neurologic symptoms were killed and their brains were frozen, sectioned, and stained for TAU.

As a result of the above experiments, it was observed in vivo that administration of PD145065 significantly reduced TAU expression in the brain of mice treated with paclitaxel or TMZ. These results show that paclitaxel may be used to inhibit the generation of or treat chemo-brain in vivo. Immunohistochemistry results are shown in FIG. 20. A significant decrease in TAU expression in the brains of mice treated with PD145065 was observed.

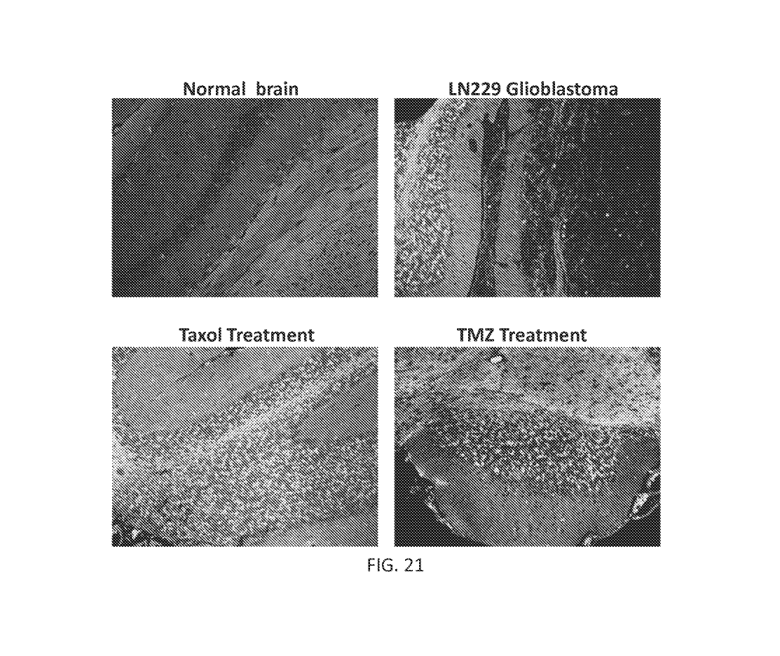

TAU expression was observed in brains from the in vivo model of chemo-brain as well as subjects with glioblastoma. As shown in FIG. 21, high expression of TAU was observed in the brains of both subjects with glioblastoma multiforme (GBM) and subjects treated with Taxol or TMZ.

Studies to evaluated prevention and/or treatment of chemo-brain by PD145065 were performed. Female athymic nude mice (NCI-nu) were used in these studies. Mice were used in these experiments in accordance with institutional guidelines when they were 14-16 weeks old. Mice were randomized as follows;

(1) Control group—mice received daily oral administration of vehicle and daily intraperitoneal injection of vehicle;

(2) PD group—mice received daily intraperitoneal injection of PD (10 mg/kg) and daily oral administration of vehicle;

(3) TMZ group—mice received daily oral administration of TMZ (7.5 mg/kg) and daily intraperitoneal injection of vehicle;

(4) Taxol group—mice received intraperitoneal injections of taxol (8 mg/kg) twice per week and daily oral administration/intraperitoneal injection of vehicle;

(5) PD+TMZ group—mice received daily oral administration of TMZ (7.5 mg/kg) and daily intraperitoneal injection of PD (10 mg/kg);

(6) PD+Taxol group—mice received intraperitoneal injection of taxol (8 mg/kg) twice per week and daily intraperitoneal injection of PD (10 mg/kg).

Mice showing weight loss, dehydration (poor skin tugor) or poor movement (poor oral intake, decreased ambulation, etc.) were defined as symptomatic mice.

Prevention and/or treatment of Chemo-brain by PD145065 was evaluated via the following protocol. Treatment continued for 12 weeks and treatment and prevention groups were defined as follows:

(1) Treatment group—mice receiving TMZ or taxol (group 3 or 4) and showing symptoms defined above for two consecutive weeks began to receive PD145065;

(2) mice receiving TMZ or taxol (group 2 or 3) without any symptoms defined above began to receive PD145065 (PD) for 2 weeks (week 11 and 12); or

(3) Prevention group—mice were receiving (PD+TMZ) or (PD+taxol) for 12 weeks (group 5 or 6).

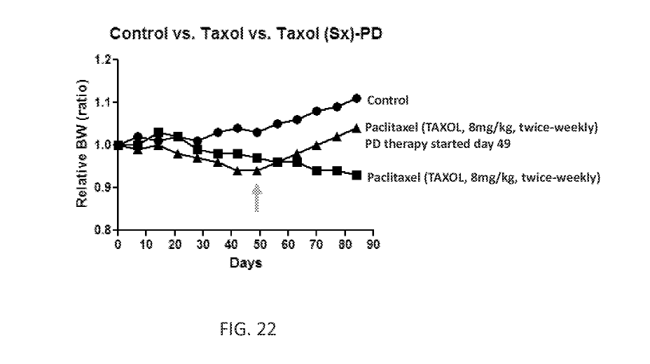

These studies involving the treatment group above demonstrated that PD145065 treated and promoted recovery from weight loss associated with paclitaxel administration. As shown in FIG. 22, PD145065 was administered to mice receiving paclitaxel beginning on day 49, and administration of the dual endothelin receptor inhibitor resulted in a reversal of the weight loss in the mice associated with the administration of paclitaxel. These studies demonstrate that a dual endothelin receptor inhibitor can be used in vivo to treat adverse side effects resulting from paclitaxel including weight loss.

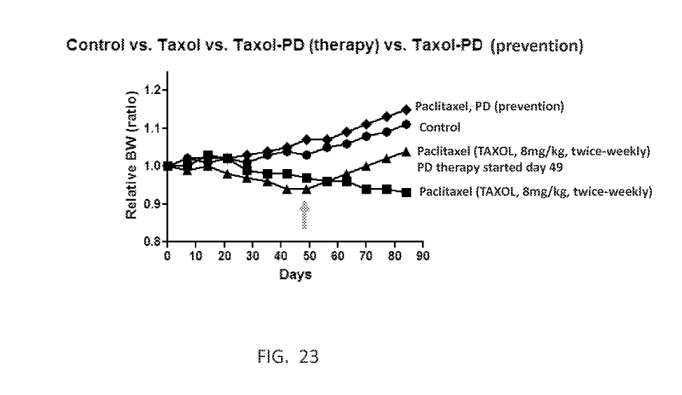

As shown in FIG. 23, co-administration of a dual endothelin receptor inhibitor (PD 145065) was sufficient to completely inhibit the weight loss associated with paclitaxel (TAXOL) administration. These studies demonstrate that a dual endothelin receptor inhibitor can be used in vivo to prevent adverse side effects resulting from paclitaxel including weight loss.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pubin. 2002/0082285
Boekhoorn et al., J. Neurosci., 26:3514-3523, 2006.
Dabir et al., J. Neurosci., 26:644-654, 2006.
De La Garza, Clin. Pharmacol., 68(7):1365-1372, 2003.
Egleton & Davis, NeuroRx., 2(1):44-53, 2005.
Fidler, Seminars in Cancer Biology, 21:107-112, 2011.
Fingl et al., In: The Pharmacological Basis of Therapeutics, 1:1, 1975.
Forman et al., J. Neurosci., 25:3539-3550, 2005.
Freireich et al., Cancer Chemother. Reports, 50:219-244, 1966.
Goedert et al., Neuron., 3:519-526, 1989.
Ishikawa et al., Proc. Natl. Acad. Sci. USA, 91(11):4892-4896, 1994.
Kim et al., Neoplasia, 13(3):286-298, 2011.
Kopke et al., J. Biol. Chem., 268:24374-24384, 1993.
Mahmood et al., J. Clin. Pharmacol., 43:692-697, 2003.
Maragakis and Rothstein, Nature Clinical Pract. Neurol., 2:679-689, 2006.
Nelson et al., Nat. Rev. Cancer, 3(2):110-116, 2003.
Remington's Pharmaceutical Sciences, 18th Ed. Mack Printing Company, 1289-1329, 1990.
Remington: The Science and Practice of Pharmacy, 21^{st} Ed. Lippincott Williams and Wilkins, 2005.
Schnyder & Huwyler, NeuroRx., 2(1):99-107, 2005.
Sengupta et al., Arch. Biochem. Biophys., 357:299-309, 1998.
Simard and Nedergaard, Neuroscience, 129:877-896, 2004.
Spencer & Verma, Proc. Natl. Acad. Sci. USA, 104(18):7594-7599, 2007.
Stamer et al., J. Cell Biol., 156:1051-1063, 2002.
Watanabe et al. Risk Analysis, 12:301-310, 1992.
Kim et al., Neoplasia, 13(3):286-298, 2011.
Schackert and Fidler, Cancer Res., 48:3478-3484, 1988.
Bolstad et al., Bioinformatics, 19:185-193, 2003.
Biometrics Research Branch, NCI, Bethesda, Md., Vol. 3.6.

1. A composition comprising: (a) a dual endothelin receptor antagonist effective to inhibit endothelin receptor A and endothelin B receptor; or (b) an endothelin receptor A antagonist and an endothelin receptor B antagonist; for use in delaying the onset or progression of a tauopathy in a subject, wherein the tauopathy is not Alzheimer's disease.

2. The composition of claim 1, wherein the composition comprises a dual endothelin receptor antagonist.

3. The composition of claim 2, wherein the dual endothelin receptor antagonist is PD145065, TAK-044, tezosentan, or bosentan.

4. The composition of claim 1, wherein the composition comprises a separate endothelin receptor A antagonist and an endothelin receptor B antagonist.

5. The composition of claim 4, wherein the endothelin receptor A antagonist is BQ123.

6. The composition of claim 4, wherein the wherein the endothelin receptor B antagonist is PD143296 or BQ788.

7. The composition of claim 1, wherein the endothelin receptor antagonist is comprised in a liposome.

8. The composition of claim 7, wherein the liposome is a CNS targeted liposome.

9. The composition of claim 1, wherein the endothelin receptor antagonist further comprises a central nervous system (CNS) targeting agent.

10. The composition of claim 9, wherein the CNS targeting agent is a CNS targeting polypeptide conjugated or fused to the endothelin receptor antagonist.

11. The composition of claim 1, wherein the tauopathy is amyotrophic lateral sclerosis/parkinsonism-dementia complex, argyrophilic grain dementia, corticobasal degeneration, creutzfeldt-Jakob disease, dementia pugilistica, diffuse neurofibrillary tangles with calcificationa, Down's syndrome, frontotemporal dementia with parkinsonism (linked to chromosome 17), Gerstmann-Straussler-Scheinker disease, Hallervorden-Spatz disease, myotonic dystrophy, Niemann-Pick disease (type C), non-Guamanian motor neuron disease with neurofibrillary tangles, Pick's disease, frontotemporal dementia and parkinsonism (FTDP), postencephalitic parkinsonism, prion protein cerebral amyloid angiopathy, progressive subcortical gliosis, progressive supranuclear palsy, subacute sclerosing panencephalitis, tangle only dementia, cognitive disorder, hypoxia, brain ischemia (cerebral ischemia), stroke, or surgical dementia, glioblastoma or glioblastoma multiforme (GBM), a traumatic brain injury (TBI), chronic encephalopathy, brain trauma, dementia pugilistica, or chemo-brain (CB).

12. A method for delaying the onset or progression of a tauopathy in a subject comprising administering to the subject:
(a) an amount of a dual endothelin receptor antagonist effective to inhibit endothelin receptor A and endothelin B receptor; or
(b) an effective amount of an endothelin receptor A antagonist and an endothelin receptor B antagonist;
wherein the tauopathy is not Alzheimer's disease.

13. The method of claim 12, wherein a dual endothelin receptor antagonist is administered to the subject.

14. The method of claim 12, wherein the dual endothelin receptor antagonist is PD145065, TAK-044, tezosentan, or bosentan.

15. The method of claim 12, wherein an effective amount of an endothelin receptor A antagonist and an endothelin receptor B antagonist are administered to the subject.

16. The method of claim 15, wherein the endothelin receptor A antagonist is BQ123.

17. The method of claim 15, wherein the endothelin receptor B antagonist is PD143296 or BQ788.

18. The method of claim 15, wherein the endothelin receptor A antagonist and the endothelin receptor B antagonist are administered in a single formulation.

19. The method of claim 15, wherein the endothelin receptor A antagonist and the endothelin receptor B antagonist are administered separately.

20. The method of claim 12, wherein the endothelin receptor antagonist is comprised in a liposome.

21. The method of claim 20, wherein the liposome is a CNS targeted liposome.

22. The method of claim 12, wherein the endothelin receptor antagonist further comprises a central nervous system (CNS) targeting agent.

23. The method of claim 22, wherein the CNS targeting agent is a polypeptide that is conjugated or fused with the endothelin receptor antagonist.

24. The method of claim 12, wherein the tauopathy is amyotrophic lateral sclerosis/parkinsonism-dementia complex, argyrophilic grain dementia, corticobasal degeneration, creutzfeldt-Jakob disease, dementia pugilistica, diffuse neurofibrillary tangles with calcificationa, Down's syndrome, frontotemporal dementia with parkinsonism (linked to chromosome 17), Gerstmann-Straussler-Scheinker disease, Hallervorden-Spatz disease, myotonic dystrophy, Niemann-Pick disease (type C), non-Guamanian motor neuron disease with neurofibrillary tangles, Pick's disease, frontotemporal dementia and parkinsonism (FTDP), postencephalitic parkinsonism, prion protein cerebral amyloid angiopathy, progressive subcortical gliosis, progressive supranuclear palsy, subacute sclerosing panencephalitis, tangle only dementia, cognitive disorder, hypoxia, brain ischemia (cerebral ischemia), stroke, or surgical dementia, glioblastoma or glioblastoma multiforme (GBM), a traumatic brain injury (TBI), chronic encephalopathy, brain trauma, dementia pugilistica, or chemo-brain (CB).

25. The method of claim 24, wherein the tauopathy is chemo-brain.

26. The method of claim 25, wherein the chemo brain results from administration of paclitaxel or temozolomide.

27. The method of claim 12, further defined as a method for delaying the onset of a tauopathy in a subject.

28. The method of claim 12, wherein the subject is at risk for developing a tauopathy.

29. The method of claim 12, wherein the subject comprises a gene mutation associated with a tauopathy or comprises a family history of tauopathy.

30. The method of claim 12, wherein the subject has reduced cognitive or memory function.

31. The method of claim 12, wherein the subject has been diagnosed with a tauopathy.

32. The method of claim 12, wherein the subject is human.

33. The method of claim 12, wherein the amount of the endothelin receptor antagonist administered to the subject is from about 10 mg/kg to about 150 mg/kg.

34. The method of claim 12, wherein the endothelin receptor antagonist is administered orally, intravenously, topically, intradermally, intraarterially, intraperitoneally, intracranially, intrathecally, intracerebroventricularly, mucosally, intrarectally (suppository), intraocularally or subcutaneously.

35. The method of claim 12, further comprising administering a second therapeutic agent to the subject.

36. The method of claim 35, wherein the second therapeutic agent is an acetylcholinesterase inhibitor or an anti-inflammatory compound.


SEQUENCE LISTING (3 sequences)

SEQ ID NO: 1 - protein, 6 aa, Artificial Sequence
  Synthetic peptide
FLFIIW

SEQ ID NO: 2 - protein, 6 aa, Artificial Sequence
  Synthetic peptide
GLDIIW

SEQ ID NO: 3 - protein, 5 aa, Artificial Sequence
  Synthetic peptide
WDPVL